(19) United States
(12) Patent Application Publication
(10) Pub. No.: US 2025/0281416 A1
(43) Pub. Date: Sep. 11, 2025

(54) COATED NANOPARTICLE AGGREGATES COMPOSED OF A HYDROLYZED METAL SALT AND METHODS OF USE THEREOF

(71) Applicants: THE BOARD OF TRUSTEES OF THE UNIVERSITY OF ILLINOIS, URBANA, IL (US); ILLINOIS INSTITUTE OF TECHNOLOGY, Chicago, IL (US)

(72) Inventors: Russell Pesavento, Chicago, IL (US); Kassapa Ellepola, Chicago, IL (US); Yuanbing Mao, Chicago, IL (US); Yuchen Zhang, Chicago, IL (US); Avin Hafedi Ghezelji, Chicago, IL (US)

(21) Appl. No.: 19/072,161

(22) Filed: Mar. 6, 2025

Related U.S. Application Data
(60) Provisional application No. 63/561,892, filed on Mar. 6, 2024.

(51) Int. Cl. A61K 9/50; A61K 9/00; A61K 33/244

(52) U.S. Cl. CPC A61K 9/5084; A61K 9/0063; A61K 33/244

(57) ABSTRACT

In accordance with the purpose(s) of the present disclosure, as embodied and broadly described herein, the disclosure relates to coated nanoparticle aggregates comprising hydrolyzed metal salt nanoparticles, wherein the aggregated hydrolyzed tetravalent metal salt nanoparticles comprise a coating of a pharmaceutically acceptable salt of a glycosaminoglycan. The coated nanoparticle aggregates are effective in reducing or preventing biofilm formation in a subject as well as reducing or preventing oxidative damage of a tissue or bone in the subject, which provides unexpected dual function properties. The coated nanoparticle aggregates exhibit good storage stability as well as enhance other products useful in preventing biofilm formation.

### CROSS-REFERENCE TO RELATED APPLICATIONS

This application claims the benefit of and priority to co-pending U.S. Provisional Patent Application No. 63/561,892, filed on Mar. 6, 2024, the contents of which are incorporated by reference herein in their entireties.

### STATEMENT REGARDING FEDERALLY SPONSORED RESEARCH OR DEVELOPMENT

This invention was made with government support under K08DE028009 awarded by the National Institutes of Health. The government has certain rights in the invention.

### BACKGROUND

Biofilm formation is a source of tissue and bone damage in patients. For example, dental caries is a multifactorial process attributed to the patient diet, genetic factors and persistent tooth-borne biofilm comprising Streptococcus mutans (S. mutans) as well as other acidogenic species. Fluoride releasing agents are effective at reducing the solubility of the tooth enamel and maintaining tooth surface integrity, however, they are limited in their ability to prevent biofilm (i.e., plaque) buildup especially in those with a diet high in fermentable carbohydrates. Thus, there remains a need for an adjunctive, cost-effective antimicrobial agent for repeated use directed toward the prevention of biofilm formation to reduce the risk of tissue and bone damage in a subject.

### SUMMARY

In accordance with the purpose(s) of the present disclosure, as embodied and broadly described herein, the disclosure relates to coated nanoparticle aggregates comprising hydrolyzed metal salt nanoparticles, wherein the aggregated hydrolyzed tetravalent metal salt nanoparticles comprise a coating of a pharmaceutically acceptable salt of a glycosaminoglycan. The coated nanoparticle aggregates are effective in reducing or preventing biofilm formation in a subject as well as reducing or preventing oxidative damage of a tissue or bone in the subject, which provides unexpected dual function properties. The coated nanoparticle aggregates exhibit good storage stability as well as enhance other products useful in preventing biofilm formation.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the synthesis and re-dispersion of CeO₂—NP-CSA.

FIGS. 2A-2C show (A) EELS spectra of CeO₂—NP-CSA, (B) low magnification STEM image of CeO₂—NP-CSA, and (C) atomic-resolution image of CeO₂—NP-CSA.

FIGS. 3A-3B show (A) UV-Vis spectra of non-formulated CeO₂—NP (3-5 nm) (dashed-line) and CeO₂—NP-CSA (solid line) (250 μM, Ce) in Milli-Q® water. The NO₃ absorbance peak (230 nm) is not present in CeO₂—NP-CSA. (B) DLS size vs intensity of non-formulated CeO₂—NP (dashed-line) vs CeO₂—NP-CSA (solid line) (Milli-Q® water). Note: The DLS spectrum of CeO₂—NP-CSA contains 30 mM KNO₃ to equal the amount of NO₃⁻ in solution from the in situ formation of CeO₂—NP.

FIGS. 4A-4D show (A) Before the addition of two equivalents of NaF to non-formulated CeO₂—NP (3-5 nm, acidic solution) and CeO₂—NP-CSA. (B) 24 h after addition of NaF to each dispersion above. (C) DLS size vs. intensity spectra of CeO₂—NP-CSA at pH 3.0 (20 mM sodium acetate buffer, blue), pH 6.0 (20 mM NaH₂PO₄ buffer, black) and, pH 10.5 (20 mM NaHCO₃ buffer, red). (D) Zeta potential of CeO₂—NP-CSA in 20 mM NaH₂PO₄ (pH 6.0).

FIGS. 5A-5B show static in vitro biofilm inhibition assay of S. mutans in UFTYE (24 h, 37° C., 5% CO₂, UFTYE+1% sucrose) utilizing a (A) microbroth dilution, dose-dependent study and crystal violet quantification on PS and (B) CFU quantification of biofilms on HA discs and orthodontic wire-PS well setup for vertical suspension of HA discs static biofilm CFU experiment (run in triplicate). Control adherent biofilm includes the average of all wells used in each row of Milli-Q©water treated wells. * indicates p<0.05, a statistically significant difference using the t test.

FIGS. 6A-6D show (A) In vitro sedimentation assay of S. mutans in PUM, pH 7.1 buffer comparing Ce-containing species (250 μM, Ce) and free CSA (the concentration present in an equal amount of CeO₂—NP-CSA). (B) Pre-incubation/mixing of all test agents in PUM, pH 7.1 for 10 minutes prior to assay initiation. * indicates a statistically significant difference (p<0.05, t-test) at 2 h. Crystal violet stained light microscopy images of planktonic growth of (C) control (Milli-Q® water treated) S. mutans in glucose supplemented UFTYE and (D) CeO₂—NP-CSA treated S. mutans.

FIG. 7 shows sedimentation assay evaluated in 20 mM Na₂HPO₄ at pH 6 (in blue) and pH 7 (in red) showed no initial clustering of S. mutans in the presence of CeO₂—NP-CSA. Exposure to Mg²⁺ (0.8 mM) initiated rapid clustering under these conditions.

FIGS. 8A-8B show (A) MTS proliferation assay (OD 490 nm) of seeded human gingival fibroblasts (HGF) cells following 48 h exposure to nanohybrid aggregates at two concentrations, and (B) A viability assay (trypan blue dye) was utilized to quantify viability of seeded hTERT-immortalized human gingival keratinocyte (TIGK) cells following 24 h exposure to the listed agents. Readings were taken in both assays A-B 1 h post exposure after washing cells to remove excess nanohybrid aggregates. No statistically significant (p<0.01, ANOVA) reduction was found under the conditions of either graph.

FIGS. 9A-9D show (A) PDI and ZP parameters of both nanohybrid aggregate formulations under similar conditions (30 mM NaH₂PO₄ buffer pH 7.0), (B) photos of stock dispersions of CeO₂—NP-CSA vs CeO₂—NP-CSA-B (30 mM), (C) low magnification LAADF image of CeO₂—NP-CSA-B, and (D) EELS spectra of CeO₂—NP-CSA-B.

FIGS. 10A-10C show in 50:50 WHS-1:MQ water, CeO₂—NP-CSA-B (A) was superior in clearance activity of S. mulans (at 250 μM, Ce) over CeO₂—NP (3-5_nm), (B) dose dependent clearance activity in sedimenting S. mutans, and (C) evidence of cell clustering effects was not observed with NaHCO₃ at the highest dose possible in the formulation of CeO₂—NP-CSA-B.

FIGS. 11A-11D show the clearance of S. mutans by CeO₂—NP-CSA-B in buffered media as a function of 12.5% WHS-1 and pH (A) pH 7 (30 mM NaH₂PO₄), (B) pH 5 (30 mM NaOAc) with DLS parameters under the same conditions (C, D). All studies were carried out at 500 μM (Ce) CeO₂—NP-CSA-B.

FIGS. 12A-12B show a 21 day efficacy study of CeO₂—NP-CSA-B (30 mM) vs. PerioMed™ (0.63% SnF₂ or 40 mM) in the reduction of (A) molar retained plaque per group (buccal surface) and (B) per molar occlusal caries in a disease induced (S. mutans) model. PerioMed™ a formulated OTC product for caries prevention was administered at recommended strength as a comparison in this study. All data was plotted as a function of the mean of the group. Statistical relevance was determined by Kruskal-Wallis non-parametric ANOVA with Dunn's post-hoc test. **p=0.0001, ****p<0.0001. (ACC Protocol #21-075)

FIGS. 13A-13D show no statistically significant change was found in response to dose dependent treatment of adult rodents with CeO₂—NP-CSA-B (30 mM) in terms of (A) rodent body weight, (B) liver enzyme activity, (C) the albumin to globulin (A/G) ratio, and the (D) relative percentages of common inflammatory cells in serum. (ACC Protocol #21-075)

FIGS. 14A-14B show (A) both single nanoceria with a starch coating vs CeO₂—NP-CSA-B demonstrating superoxide dismutase (SOD) activity. (B) Plate based assay (SOD kit, Millipore Sigma) in 33:67 WHS/Assay buffer at 10 μM Ce demonstrating activity compared to SOD enzyme itself (red) (IRB Protocol #2023-0562).

FIGS. 15A-15B show (A) both single nanoceria with a starch coating vs CeO₂—NP-CSA-B demonstrating catalase (CAT) activity. (B) Plate based assay (Amplex Red Assay, Invitrogen) was used to screen CAT activity in 33:67 MCM/TRIS buffer pH 7.0 at 150 μM Ce demonstrating activity compared to CAT enzyme itself (red). (MCM: Mucin containing medium)

FIG. 16 shows several different polymer coated cerium nanoparticles that were evaluated with respect to SOD and CAT activity.

FIGS. 17A-17B show an inhibition comparison of different nanoceria formulations going from TRIS buffer (pH 7.0) (A) to a mixture of (33%) WHS (SOD activity) and (B) a mixture of (33%) MCM (CAT activity). The most effective catalysts for each assay were evaluated.

FIG. 18 shows the anti-oxidant activity of CeO₂—NP-CSA-B with HGF cells.

FIG. 19 shows human gingival fibroblasts (HGF) were exposed to hydrogen peroxide (and DCF-DA) as described above (FIG. 18) and then were incubated for 2 hours with MTS and the optical density was measured with a plate reader spectrophotometer in order to assess viability following ROS induction. CeO₂—NP-CSA-B outcompeted a known and FDA approved antioxidant (N-acetyl cysteine) in protecting HGF cells from ROS damage. Chronic oxidative damage is linked to common oral pathologies as described previously. *p<0.05, **p<0.01—Two-way ANOVA.

FIG. 20 shows HGFs were seeded into a 96-well tissue culture plate at a density of 15 k cells/well and left to adhere overnight. ROS was induced using 500 μM of H₂O₂, and additional treatments (as specified) were added to the cells and incubated for 1 hour. Following 1 hour treatment, cells were washed, stained with DCF-DA, rinsed and then the MTS assay was performed. The optical density (OD) was read at 490 nm following 1 hour of incubation with MTS reagent. NAC=N-acetyl cysteine, an FDA approved anti-oxidant. **p<0.01, ****p<0.0001—Two-way ANOVA.

FIGS. 21A-21B show the storage of CeO₂—NP-CSA-B in Biotene™ for 5 days at room temperature.

FIG. 22 shows the clearance activity of S. mutans UA159 by CeO₂—NP-CSA-B in a Biotene™-WHS-1 mixture.

FIG. 23 shows the catalase activity of CeO₂—NP-CSA-B following storage in Biotene™.

FIG. 24 shows the catalase activity of CeO₂—NP-CSA-B immediately upon addition to 33% Biotene™/tris buffer.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Streptococcus mutans, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, amelioration or palliation of the disease state, and remission (partial or total) whether detectable or undetectable.

As used herein, the term “prevent” or “preventing” refers to precluding, averting, obviating, forestalling, stopping, or hindering something from happening, especially by advance action.

As used herein, the term “reduce” or “reducing” refers to deceasing the degree or amount of an event. For example, reducing biofilm formation can mean lower the amount of biofilm formation when using the compositions described herein when compared to the amount of biofilm formation when the compositions described herein are not used.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Coated Nanoparticle Aggregates and Methods of Making and Using the Same

In one aspect, disclosed herein are coated nanoparticle aggregates comprising aggregated hydrolyzed tetravalent metal salt nanoparticles, wherein the aggregated hydrolyzed tetravalent metal salt nanoparticles comprise a coating of a pharmaceutically acceptable salt of a glycosaminoglycan. The coated nanoparticle aggregates are effective in reducing or preventing biofilm formation in a subject.

The phrase “aggregated hydrolyzed tetravalent metal salt nanoparticles” is defined herein as a collection or plurality of nanoparticles that are agglomerated or adhered to one another. The aggregated hydrolyzed tetravalent metal salt nanoparticles can be prepared initially then subsequently coated with the pharmaceutically acceptable salt of a glycosaminoglycan. During the coating process, the aggregated hydrolyzed tetravalent metal salt nanoparticles as well as individual nanoparticles are coated and adhered to one another by the pharmaceutically acceptable salt of the glycosaminoglycan. The coating process can partially or completely coat the aggregated hydrolyzed tetravalent metal salt nanoparticles and any free nanoparticles that may be present. The Examples provide non-limiting procedures for making the coated nanoparticle aggregates described herein. In one aspect, the aggregated hydrolyzed tetravalent metal salt nanoparticles have an average microscopic diameter as determined by TEM of about 20 nm to about 500 nm, or about 20 nm, 50 nm, 100 nm, 150 nm, 200 nm, 250 nm, 300 nm, 350 nm, 400 nm, 450 nm, or 500 nm, where any value can be a lower and upper endpoint of a range (e.g., 100 nm to 250 nm).

As demonstrated herein, the coated nanoparticle aggregates are effective in inhibiting the initiation or early stages of in vitro biofilm formation to significantly reduce adherent biofilm formation. Biofilm formation is a source of tissue and bone damage in patients. For example, dental caries is a multifactorial process attributed to the patient diet, genetic factors and persistent tooth-borne biofilm comprising Streptococcus mutans as well as other acidogenic species. In one aspect, the coated nanoparticle aggregates when administered to a subject can clear bacteria such as Streptococcus mutans so that biofilm formation cannot occur on the teeth and gums of the subject. The coated nanoparticle aggregates are effective in reducing or preventing biofilm formation, which in turn, reduces or prevents the onset of infection. In other aspects, the coated nanoparticle aggregates described herein can be used to reduce or prevent the formation of plaque, gingivitis or periodontal disease.

The coated nanoparticle aggregates described herein also possess antioxidant properties. Catalase (CAT) and superoxide dismutase (SOD) in whole human saliva help remove reactive oxygen species (ROS). For example, chronic hyposalivation has been associated with oral pathologies such as, for example, oral lichen planus, oral squamous cell carcinoma, and recurrent aphthous stomatitis, which is partly attributed to prolonged exposure to ROS. The coated nanoparticle aggregates described herein possess an effective and superior combination of CAT and SOD-like activity.

The coated nanoparticle aggregates described herein possess dual active properties of (1) reducing or preventing biofilm formation and (2) reduce or prevent damage caused by ROS. These unique properties provide numerous health benefits, particularly when addressing oral pathologies and diseases. In one aspect, the coated nanoparticle aggregates can reduce or preventing the formation of a plaque or a dental caries in a subject, which can be formed by a biofilm.

In another aspect, the coated nanoparticle aggregates can reduce or prevent oxidative damage or stress of a tissue or bone in a subject. In one aspect, the coated nanoparticle aggregates can reduce or prevent oxidative damage in or around the oral cavity of the subject. For example, the coated nanoparticle aggregates described can be used to reduce, treat, or prevent oral mucositis. Oral mucositis is a debilitating pathology that is a common side effect when a subject to exposed to radiation therapy in the neck and head region to treat cancer. Oral mucositis has an etiology of high localized ROS. The coated nanoparticle aggregates with their antioxidant properties described herein can be used as an effective treatment for oral mucositis and other mucosal tissue susceptible to ROS.

The coated nanoparticle aggregates are effective in reducing or preventing biofilm formation in a subject. However, the coated nanoparticle aggregates can be applied to a substrate where there is the likelihood of biofilm formation. In one aspect, the coated nanoparticle aggregates can be applied to a coating on an implantable device such as, for example, catheters (e.g., urinary or vascular), cannulas, tubing, sutures, an artificial heart valve, a graft (e.g., vascular graft), a stent (e.g., coronary stent), or circuitry (e.g., extracorporeal membrane oxygenation circuit).

In other aspects, the coated nanoparticle aggregates described herein can be used to produce a component to fabricate a medical device. For example, the coated nanoparticle aggregates described herein can be used to produce cannulas, catheters, heart valves, endovascular stents, and joint prostheses, and other medical devices. In one aspect, the coated nanoparticle aggregates described herein can be formulated in a solvent alone or in combination with one or more additional components then subsequently poured into a mold. The solvent then can be removed to produce a molded article composed of the coated nanoparticle aggregates.

As will be discussed in detail below, the coated nanoparticle aggregates described herein can be formulated as a number of different pharmaceutical applications depending upon the application and mode of administration. In one aspect, the coated nanoparticle aggregates described herein can be formulated as an oral or topical composition. For example, the pharmaceutical composition can be a mouthwash, a beverage, a gel, a dissolvable strip, a varnish, or a toothpaste composed of the coated nanoparticle aggregates described herein.

In certain aspects, the coated nanoparticle aggregates described herein can be formulated with other bioactive agents. In one aspect, the pharmaceutical composition includes the coated nanoparticle aggregates described herein and a bioactive agent including, but not limited to, an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof.

In one aspect, the coated nanoparticle aggregates described herein can be formulated with a mouthwash to promote oral healthcare. For example, the coated nanoparticle aggregates described herein can be added to Biotene™, which is a commercially available mouthwash or saliva substitute for those with chronic hyposalivation. In another aspect, the coated nanoparticle aggregates described herein can be used in combination with sodium fluoride or other fluoride containing agents that are utilized commonly in oral health products.

The coated nanoparticle aggregates described herein include hydrolyzed metal salt nanoparticles coated with pharmaceutically acceptable salt of a glycosaminoglycan. Each component of the coated nanoparticle aggregates and methods for making the same are provided below.

### Hydrolyzed Metal Salts

The coated nanoparticle aggregates described herein include hydrolyzed metal salts. In one aspect, the hydrolyzed metal salt is a tetravalent metal salt. Tetravalent metal salt is any such metal salt (e.g., nitrate, halide, sulfate, etc.) that is in the +4 oxidation sate. In some embodiments the hydrolyzed tetravalent metal salt can be Ce(IV), Zr(IV), Hf(IV), or Ti(IV). The hydrolysis products of tetravalent metals salts can exist as either mononuclear complexes, oligomers/polymers, nanoparticles or a combination thereof. In some embodiments, the hydrolysis products of tetravalent metals salts can exist as nanoparticles. In some embodiments, the nanoparticles can have an average hydrodynamic radius of approximately 25 nm, 20 nm, 15 nm, 10 nm, 9 nm, 8 nm, 7 nm, 6 nm, 5 nm, 4 nm, 3 nm, 2 nm, 1 nm, or smaller, where any value can be a lower and upper endpoint of a range (e.g., 2 nm to 10 nm).

In one aspect, the hydrolyzed tetravalent metal salt is an oxide. For example, the hydrolyzed tetravalent metal salt can be nanoparticles of CeO₂ (referred to herein as CeO₂—NP). In one embodiment, the CeO₂—NP are prepared with the use of ceric ammonium nitrate (CAN) or a cerium (IV) sulfate sulfuric acid complex at low concentrations in accordance with literature methods. In another aspect, the hydrolyzed metal salt can be Fe⁺² salt or Fe⁺³ salt (e.g., ferric or ferrous oxide).

In one aspect, the aggregated hydrolyzed metal salt nanoparticles can be produced by solubilizing the metal salt in water. The hydrolyzed metal salt nanoparticles can be a hydroxide, an oxide, or a combination thereof. Non-limiting methods for producing the hydrolyzed metal salt nanoparticles is provided in the Examples.

In some aspects, the hydrolyzed metal salts described herein can be mixed with a metal salt, such as a divalent metal salt in some formulations. Suitable additional metal salts that can be added to the hydrolyzed tetravalent metal salts described herein, include but are not limited to salts of Ca(II), Sr(II), Ba(II), Zn(II), Cu (II), Be(II), Ni(II), Fe(II), Co(II), Mn(II), Cr(II), V(II), Ti(II), Sc(II), Cd(II), Hg(II), and cacodylic acid (As) sodium salt.

In some aspects, the hydrolyzed metal salts described herein can be mixed with a metal salt, such as a monovalent metal salt in some formulations. Suitable additional metal salts that can be added to the hydrolyzed tetravalent metal salts described herein, include but are not limited to, salts of Group I elements of the Periodic Table of Elements, Cu(I), Ag(I), and Au(I).

In other aspects, mixtures and formulations of the aggregated hydrolyzed metal salt nanoparticles can contain a suitable monovalent metal containing compounds. Suitable monovalent metals include, but are not limited to, Group I elements of the Periodic Table of Elements, Cu(I), Ag(I), and Au(I) as well as any salt thereof.

### Glycosaminoulycan

Glycosaminoglycans (GAGs) are long, linear polysaccharides consisting of repeating disaccharide units (i.e. two-sugar units). The repeating two-sugar unit consists of a uronic sugar and an amino sugar, except in the case of the sulfated glycosaminoglycan keratan, where, in place of the uronic sugar there is a galactose unit. In one aspect, the glycosaminoglycan is hyaluronic acid or heparin.

In one aspect, the glycosaminoglycan is a sulfated glycosaminoglycan, which is a glycosaminoglycan (GAG) having at least one sulfate group. In one aspect, the sulfated glycosaminoglycan is chondroitin sulfate, dermatan sulfate, keratan sulfate, or heparan sulfate.

In one aspect, the sulfated glycosaminoglycan is a sulfated hyaluronan or the pharmaceutically acceptable salt or ester thereof. In one aspect, the sulfated hyaluronan has a degree of sulfation from 0.1 to 4.0 per disaccharide unit. In another aspect, the sulfated hyaluronan has a degree of sulfation from 0.1, 0.5, 1.0, 1.5, 2.0, 2.5, 3.0, 3.1, 3.2, 3.3, 3.4, 3.5, 3.6, 3.7, 3.8, 3.9, or 4.0 per disaccharide unit, where any value can be a lower and upper end-point of a range (e.g., 3.0 to 4.0, 3.2 to 3.8, etc.).

In another aspect, the average molecular weight of the sulfated hyaluronan is less than 1,000 kDa, less than 900 kDa, less than 800 kDa, less than 700 kDa, less than 600 kDa, less than 500 kDa, less than 400 kDa, less than 300 kDa, less than 200 kDa, less than 100 kDa, less than 50 kDa, less than 25 kDa, less than 10 kDa, or less than 5 kDa. In another aspect, the sulfated hyaluronan has an average molecular size from 0.5 kDa to less than 50 kDa, 2 Da to 20 kDa, or 3 kDa to 10 kDa. In a further aspect, the sulfated hyaluronan has an average molecular size from 0.5 kDa to 10 kDa or 1 kDa to 10 kDa. Depending upon reaction conditions, one or more different hydroxyl groups present in the low molecular hyaluronan or hyaluronan oligosaccharide can be sulfated. In one aspect, the primary C-6 hydroxyl proton of the N-acetyl-glucosamine residue of the low molecular hyaluronan or hyaluronan oligosaccharide is sulfated. In another aspect, the primary C-6 hydroxyl proton of the N-acetyl-glucosamine residue of hyaluronan and at least one C-2 hydroxyl proton or C-3 hydroxyl proton of a uronic acid residue or at least one C-4 hydroxyl proton of an N-acetyl-glucosamine residue is substituted with a sulfate group. In another aspect, the primary C-6 hydroxyl proton of the N-acetyl-glucosamine residue of the low molecular hyaluronan or hyaluronan oligosaccharide and at least one C-2 hydroxyl proton and C-3 hydroxyl proton of a uronic acid residue and at least one C-4 hydroxyl proton of an N-acetyl-glucosamine residue is substituted with a sulfate group. In another aspect, 0.001%, 0.01%, 0.1%, 1%, 5%, 10%, 20%, 30%, 40%, 50%, 60%, 70%, 80%, 90%, 95%, or less than 100%, or any range thereof of hydroxyl protons present on the low molecular hyaluronan or hyaluronan oligosaccharide can be deprotonated and subsequently sulfated.

In another aspect, the sulfated hyaluronan has (1) 90% of the primary C-6 hydroxyl protons of the N-acetyl-glucosamine residue of the sulfated hyaluronan are substituted with a sulfate group, (2) a degree of sulfation from 3.0 to 4.0, and (3) an average molecular weight from 1 kDa to 10 kDa. In another aspect, sulfated hyaluronan has (1) 100% of the primary C-6 hydroxyl protons of the N-acetyl-glucosamine residue of the sulfated hyaluronan are substituted with a sulfate group, (2) a degree of sulfation from 3.0 to 4.0, and (3) an average molecular weight from 1 kDa to 10 kDa.

In one aspect, the glycosaminoglycan is chondroitin sulfate A. Chondroitin sulfate A is an acidic mucopolysaccharide found in cartilage, skin, cornea, and umbilical cord. It is composed of alternating N-acetylgalactosamine, D-glucuronic acid, and sulfate residues in equimolar quantities where carbon 4 of the N-acetylgalactosamines is sulfated. In one aspect, the glycosaminoglycan is a sodium salt of chondroitin sulfate as provided by Alfa Aesar.

In another aspect, the glycosaminoglycan is chondroitin sulfate B. Chondroitin sulfate B can be purchased and used directly to produce the coated nanoparticle aggregates or, in the alternative, can be produced in situ using a buffer such as, for Example, NaHCO₃. The Examples provide non-limiting procedures for producing coated nanoparticle aggregates with chondroitin sulfate B.

The coated nanoparticle aggregates can be produced by mixing the aggregated hydrolyzed metal salt nanoparticles with the pharmaceutically acceptable salt of a glycosaminoglycan. In one aspect, the aggregated hydrolyzed metal salt nanoparticles are mixed with the pharmaceutically acceptable salt of the glycosaminoglycan in water to produce a precipitate composed of the coated nanoparticle aggregates. The coated nanoparticle aggregates can be isolated and used as is or, in the alternative, further processed. For example, the coated nanoparticle aggregates can undergo buffer exchange (e.g., NaHCO₃). The Examples provide non-limiting procedures for producing the coated nanoparticle aggregates.

The coated nanoparticle aggregates are stable in water and possess a long shelf-life (e.g., greater than six months), which makes them useful in the formulation of numerous healthcare products.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, Iinolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Aspects

Aspect 1. A coated nanoparticle aggregate comprising aggregated hydrolyzed tetravalent metal salt nanoparticles, wherein the aggregated hydrolyzed tetravalent metal salt nanoparticles comprise a coating of a pharmaceutically acceptable salt of a glycosaminoglycan.

Aspect 2. The coated nanoparticle aggregate of aspect 1, wherein the hydrolyzed tetravalent metal salt comprises a hydrolysis product of a Ce(IV) salt, a Zr(IV) salt, a Hf(IV) salt, or a Ti(IV) salt.

Aspect 3. The coated nanoparticle aggregate of aspect 1, wherein the hydrolyzed tetravalent metal salt comprises a hydrolysis product of Ce(IV) salt.

Aspect 4. The coated nanoparticle aggregate of aspect 1, wherein the hydrolyzed tetravalent metal salt comprises CeO₂.

Aspect 5. The coated nanoparticle aggregate of aspect 1, wherein the aggregated hydrolyzed tetravalent metal salt nanoparticles have an average microscopic diameter of about 20 nm to about 500 nm.

Aspect 6. The coated nanoparticle aggregate of aspect 1, wherein the glycosaminoglycan comprises a sulfated glycosaminoglycan.

Aspect 7. The coated nanoparticle aggregate of aspect 1, wherein the glycosaminoglycan comprises heparin, heparan sulfate, chondroitin sulfate, keratan sulfate, hyaluronic acid, or any combination thereof.

Aspect 8. The coated nanoparticle aggregate of aspect 1, wherein the coating comprises the sodium salt of chondroitin sulfate

Aspect 9. The coated nanoparticle aggregate of aspect 1, wherein the coated nanoparticles have a zeta potential of about −15 mV to about −40 mV.

Aspect 10. The coated nanoparticle aggregate of aspect 1, wherein the coated nanoparticles have a polydispersion index of about 0.2 to about 0.5.

Aspect 11. The coated nanoparticle aggregate of aspect 1, wherein the coated nanoparticles further comprise one or more additional metal salts of Ca(II), Sr(II), Ba(II), Zn(II), Cu (II), Be(II), Ni(II), Fe(II), Co(II), Mn(II), Cr(II), V(II), Ti(II), Sc(II), Cd(II), Hg(II), cacodylic acid (As) sodium salt, or any combination thereof.

Aspect 12. The coated nanoparticle aggregate of aspect 1, wherein the coated nanoparticle aggregate is produced by the process comprising admixing the aggregated hydrolyzed tetravalent metal salt nanoparticles with the pharmaceutically acceptable salt of chondroitin sulfate in a solvent.

Aspect 13. A pharmaceutical composition comprising the coated nanoparticle aggregate of aspect 1 and a pharmaceutically acceptable carrier.

Aspect 14. The composition of aspect 13, wherein the composition comprises an oral or topical composition.

Aspect 15. The composition of aspect 13, wherein the composition comprises a mouthwash, a beverage, a gel, a dissolvable strip, a varnish, or a toothpaste

Aspect 16. The composition of aspect 13, wherein the composition comprises a mouthwash comprising sodium monofluorophosphate, sodium fluoride, or a combination thereof.

Aspect 17. The composition of aspect 13, wherein the composition can be stored at room temperature for at least 6 months.

Aspect 18. A method for reducing or preventing the formation of a biofilm in a subject, the method comprising administering to the subject the coated nanoparticle aggregate of aspect 1.

Aspect 19. The method of aspect 18, wherein the coated nanoparticle aggregate reduces or prevents the formation of the biofilm in an oral cavity of the subject.

Aspect 20. The method of aspect 18, wherein the coated nanoparticle aggregate is administered to the subject orally or topically.

Aspect 21. A method for reducing or preventing oxidative damage or stress of a tissue in a subject, the method comprising administering to the subject the coated nanoparticle aggregate of aspect 1.

Aspect 22. The method of aspect 21, wherein the coated nanoparticle aggregate reduces or prevents oxidative damage in or around the oral cavity of the subject.

Aspect 23. The method of aspect 21, wherein the nanoparticles are administered to the subject orally or topically.

Aspect 24. A method for reducing or preventing the formation of a plaque, dental caries, or a periodontal disease in a subject, the method comprising administering to the subject the coated nanoparticle aggregate of aspect 1.

Aspect 25. A method for treating or preventing skin cancer or oral cancer in a subject, the method comprising administering to the subject the coated nanoparticle aggregate of aspect 1.

Aspect 26. A medical device comprising one or more surfaces partially or completely coated with the coated nanoparticle aggregate of aspect 1.

Aspect 27. The device of aspect 26, wherein the device comprises a catheter, a heart valve, an implant, a surgical pin, a contact lens, a cannula, a tubing, a suture, a graft, a stent, or circuitry.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Example 1

### Experimental Section

### Materials

All chemical reagents were purchased commercially and utilized without further purification. Ultrapure Milli-Q® water was used to prepare all samples for synthetic preparation, Dynamic Light Scattering (DLS) and Zeta Potential (ZP) measurements, Scanning Transmission Electron Microscopy and Electron Energy Loss Spectroscopy (STEM/EELS) data collection and UV-Vis Spectroscopy. Ceric nitrate (1.0 N aqueous solution) was purchased from GFS Chemicals (Columbus, Ohio). Chondroitin sulfate A, sodium salt (90%) was purchased from Alfa Aesar (Ward Hill, MA). Dextrose, sucrose, KNO₃, NaHCO₃, NaOH were all purchased from Fisher Scientific (Fair Lawn, New Jersey). Disposable BrandTech PMMA cuvettes (Cole-Parmer, Vernon Hills, IL) and folded capillary cells (Malvern Instruments, Worcestershire, UK) were used for size and zeta potential measurements. Streptococcus mutans (S. mutans) UA159 culture was prepared from frozen glycerol stock and was maintained in brain heart infusion (BHI; Hardy Diagnostics, Santa Maria, CA). Solid media were prepared by adding 1.5% (w/v) Bacto agar (IBI Scientific, Peosta, IA). Unless otherwise stated, cultures were maintained in an aerobic chamber at 37° C. with inclusion of 5% CO₂. For the biofilm experiments, ultra-filtered (membrane with a 10-kDa molecular mass cutoff) tryptone yeast extract medium (UFTYE; pH 7.0) containing 1% (w/v) sucrose was used. All pH measurements were carried out on a benchtop Fisherbrand Accumet AB200 pH and conductivity meter following acidic, neutral and basic pH calibration.

Synthesis of CeO₂—NP-CSA. A 10 mL, 10 mM dispersion of non-formulated CeO₂—NP (3-5 nm) was prepared from the hydrolysis of 1 N H₂[Ce(NO₃)₆] and allowed to stand for 24 h at room temperature. To this dispersion, 105 μL of a 10% (wt.) solution of chondroitin sulfate A, sodium salt (Acros) was added which caused the formation of an immediate off-white precipitate. This mixture was vortexed for 30-60 seconds and allowed to stand for approximately 5-10 min. Once the precipitate was separated from the parent mixture, the content was centrifuged for 1 min (3900 rpm, 4° C.). The supernatant was decanted from the precipitate, and 3-5 mL of Milli-Q® water was added to the precipitate, re-dispersed with gentle vortexing and the supernatant decanted (4×). Following removal of the supernatant from the final wash, a fresh 10 mM NaHCO₃ (8.0 mL) solution adjusted to pH 9.5 (with 1 M NaOH) was added to the off-white precipitate and was gently vortexed intermittently over the next several hours. The mixture was allowed to stand overnight and the precipitate was dispersed with gentle vortexing the next day. The entire dispersion (8.0 mL) was then concentrated down to a yellow tinted filtrate with the use of an Amicon filter (15 mL volume, 50 kDa MW cut-off) via centrifuging for 12-15 minutes (3900 rpm, 4° C.). Milli-Q® water (2-3 mL) was added to the filtrate, gently vortexed and concentrated as described above. The washing-centrifuging process was repeated (3×). Following the last wash, the filtrate was re-dispersed in approximately 2.5 mL of Milli-Q® water to an estimated≈30 mM (Ce). The absorbance peak at 290 nm (UV-Vis Spectroscopy) was used to estimate the amount of CeO₂—NP in this dispersion, and adjusted accordingly with Milli-Q® water. ICP-OES (Ce) analysis confirmed equivalent Ce content in both CeO₂—NP (3-5 nm) and CeO₂—NP-CSA with a total yield of 84% (recovery of Ce) for the CeO₂—NP-CSA preparation. The Blyscan assay was used to estimate the CSA content in CeO₂—NP-CSA. At a concentration of 30 mM (Ce) CeO₂—NP-CSA (over 5.0 mg/mL CeO₂—NP), the CSA concentration is estimated at ≈2.0 mg/mL from the Blyscan assay. For comparison, 2 mg/mL free CSA solution was found to have the same S content (in ppm) by ICP-OES as in 30 mM (Ce) CeO₂—NP-CSA. CeO₂—NP-CSA was stored at 4° C. for several months with only minimal sedimentation.

### Characterization of CeO₂—NP-CSA.

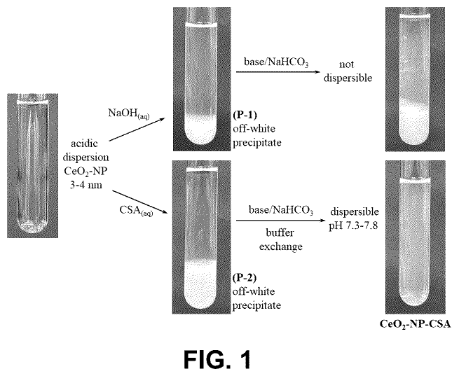

X-ray Diffraction Studies (XRD). Both P-1, P-2 (FIG. 1) were analyzed via XRD. The crystal phase of the prepared samples was determined using a powder X-ray diffractometer (Bruker AXS D2) with Cu K_{α} radiation (λ=1.5406 Å) in the 2θ range from 10° to 80° with the scanning step size of 0.01° and step time of 0.05 sec. XRD data was plotted on Origin (OriginLab Corporation, North Hampton, MA).

Fourier Transform Infrared Spectroscopy (FT-IR). Duplicate samples of non-formulated CeO₂—NP (3-5 nm, 10 mM Ce) were prepared in double-distilled water and stored at room temperature. Immediate precipitation (P-1, P-2) was observed upon addition of either 1 M NaOH (325 μL) or 10% wt. CSA (50 μL) to non-formulated CeO₂—NP (respectively) (FIG. 1). The off-white precipitates (P-1, P-2) were washed (4×) with double-distilled water (1-2 mL) with the supernatant decanted off upon the final rinsing. Precipitates were allowed to dry in air >4 days and then stored under reduced pressure. The dried materials were ground with mortar and pestle into a fine, crystalline-like powder. The FT-IR (ATR) absorbance spectra of each P-1 and P-2 were obtained on a Perkin Elmer Spectrum Two FT-IR Spectrophotometer.

Inductively Coupled Plasma Optical Emission Spectroscopy (ICP-OES). ICP-OES sample analysis was carried out at the University of Illinois Urbana-Champaign (UIUC) Microanalysis Laboratories (Champaign, Illinois). Ce-analysis was carried out by first estimating the concentration by the UV-Vis absorbance at 290 nm and then carrying out a series of dilutions in Milli-Q® water to an estimated concentration of 140 ppm (1 mM Ce). All samples (e.g., CeO₂—NP, CeO₂—NP-CSA) were digested with a mixture of 2.5 mL hydrogen peroxide (30%), 5 mL sulfuric acid (97%), and 2.5 mL Milli-Q® water for cerium analysis and 10 mL nitric acid (70%) for sulfur analysis. The sample solutions were then subjected to the CEM MARS6 microwave digestion system utilizing the following parameters: Power 700-1800 W, Temperature 240° C., Ramp Time 30 min, Hold Time 15 min. Following complete digestion, the samples were diluted to a 25 mL final volume. Standard calibration curves for both Ce and S were prepared in 2% nitric acid. Samples and standards were analyzed on a Perkin Elmer Optima 8300 ICP-OES instrument with the following parameters: Plasma (Argon) gas flow: 10 L/min; Auxiliary gas flow: 0.20 L/min; Nebulizer gas flow 0.8 L/min; RF Power 1500 W and a pump flow 1.5 mL/min. The emission wavelength used with 418.66 nm for Ce and 181.975 nm for S, both using axial view. Ce and S concentrations were calculated via a linear regression (R²>0.999). A QC standard was run after the samples, which was within ±10% of the expected concentration.

Gel Permeation Chromatography (GPC). GPC was utilized to estimate the CSA (Acros) molecular weight and dispersity. CSA powder was dissolved in 1×PBS at a concentration of ˜5 mg/mL for several hours under periodic mixing, followed by filtration through a 0.22 μm nylon syringe filter into an HPLC vial for analysis. Sample analysis was carried out on a Tosoh EcoSEC system with refractive index and UV detection at ambient temperature. The mobile phase (1× PBS) flow rate was held constant at 1 mL/min and the stationary phase was a series of 3 Agilent PL Aquagel-OH size exclusion columns (xx-6820, xx-6840, xx-6860). A series of 8 PEG/PEO standards (Agilent Easivial) were analyzed prior to sample analysis for molar mass calibration. The weight average molecular weight (Mw) of the as-prepared sample was equivalent to ≈47,000 g/mol (PEG) under similar conditions.

Blyscan Assay. The Blyscan assay (Biocolor Ltd, Antrim, UK) was carried out to determine the concentration of CSA in the formulation of CeO₂—NP-CSA. Following serial dilutions of the unknown samples (non-formulated CeO₂—NP, 3-5 nm), CeO₂—NP-CSA) the concentration of CSA was estimated from the best fit standard curve (linear least squares fit) plot. No CSA was found in CeO₂—NP as a control measure.

High Resolution Scanning Transmission Electron Microscopy (STEM) and Electron Energy Loss Spectroscopy (EELS). CeO₂—NP-CSA was diluted to 5 mM (Ce) in Milli-Q® water and stored at cold temperatures prior to measurements. The atomic-resolution STEM and EELS maps were collected using the JEOL ARM2000F aberration corrected STEM with a cold-field emission gun operated at an acceleration voltage of 200 kV and 80 kV. EELS for sample CeO₂—NP-CSA was taken at 80 kV. Low-angle annular dark field (LAADF) imaging was also utilized. The LAADF images were acquired using an annular dark-field detector with a collection angle ranging from 30 to 120 mrad and a probe current of 19 pA. For EELS measurement, the probe convergence semi-angle was set to 17.8 mrad, which yields a probe size of 1 Å and a probe current of 62 pA. EELS characterization was conducted using the post-column Gatan Continuum GIF spectrometer using a collection semi angle of 53.4 mrad. Samples were encapsulated in graphene liquid cells with aqueous media as previously reported. ²⁵⁻²⁸ STEM images were recorded via Gatan Digital Micrograph as part of the Gatan Microscopy Suite (GMS, Gatan Inc., Pleasanton, CA) and the EELS spectrum was graphed via Kaleidagraph (Synergy Software Inc., Reading, PA).

UV-Vis Spectroscopy. All UV-Vis spectra were obtained utilizing a NanoDrop One^{C} Spectrophotometer (Thermo Scientific). Absorbance of dispersions of non-formulated CeO₂—NP (3-5 nm) and CeO₂—NP-CSA (in Milli-Q® water) were used to estimate the concentration of CeO₂—NP (290 nm) in disposable UV-Cuvettes (BrandTech). Quantification of sedimentation assays was measured by absorbance (OD₆₀₀) in various buffered media.

Dynamic Light Scattering (DLS) and Zeta Potential (ZP) Measurements. All size vs intensity data was collected at the University of Illinois Chicago (UIC) Nanotechnology Core Facility (NCF) utilizing a Malvern Zetasizer ZSP. The average hydrodynamic diameter (nm) of each sample was determined via the function of intensity weighted distribution of the Malvern software program. All samples analyzed by DLS were dispersed in Milli-Q® water (18.2 MΩ) based buffers and measured in capillary folded disposable folded capillary cells (Malvern). ZP measurements were made at the time of the size vs intensity experiments described above under similar conditions, with an additional size vs intensity replicate measurement following the zeta potential measurements to insure sample consistency. All hydrodynamic diameter and ZP measurements were carried out with a minimum of three replicate runs and reported as an average and standard deviation of these replicate measurements.

Static Biofilm Inhibition Assays. Overnight grown S. mutans UA159 were transferred into UFTYE medium²², supplemented with 1% glucose and grown until OD₆₀₀˜0.5, diluted 1:100 in UFTYE containing 1% sucrose and added to the wells of a 96-well microtiter plate (CELLTREAT). The bacterial cells were treated with Ce-containing agents at several concentrations [1000 μM, 500 μM, 250 μM, 125 μM (Ce)] using the microdilution method. The polystyrene (PS) plate was incubated statically at 37° C. for 24 h with inclusion of 5% CO₂. Biofilms were quantified by staining with 0.1% crystal violet and washed to remove unbound stain. The plate was air dried and de-stained using a mixture of ethanol:acetone (4:1), and subsequently quantitated using a spectrophotometer at 595 nm.²⁹ Orthodontic wire-PS well setup was used for vertical suspension of hydroxyapatite (HA) discs for the static biofilm CFU experiment. S. mutans cultures of OD₆₀₀˜0.5 were diluted 1:100 in UFTYE containing 1% sucrose and added to the wells of a 24-well microtiter plate containing the HA disc with CeO₂—NP-CSA [250 μM (Ce)] and CSA supplemented at a concentration estimated to be present at 250 μM (Ce) and incubated using conditions described above. Following 24 h incubation, HA discs were washed and placed in a test tube containing phosphate buffered saline (PBS) and sonicated on ice for 10 seconds at an amplitude of 20% using a sonicator (Qsonica) followed by vigorous vortexing. The cell suspension was serially diluted and plated out on BHI agar plates for S. mutans enumeration. The average number of colony forming units (CFU) per disc was determined.

Biofilm Dispersal Assay. S. mutans UA159 biofilms were grown in 96-well plates in the presence of UFTYE containing 1% sucrose for 24 h at 37° C. under 5% CO₂. Established biofilms were exposed to non-formulated CeO₂—NP (3-5 nm), CeO₂—NP-CSA and Ce(NO₃)·6H₂O at a final concentration of 2 mM (Ce) for 10 min. The test agents were removed from the wells and the remaining biofilm was washed once with 200 μL of PBS to remove non-adhered cells. A volume of 200 μL of 0.1 mg/mL XTT (2,3-Bis-(2-Methoxy-4-Nitro-5-Sulfophenyl)-2H-Tetrazolium-5-Carboxanilide; VWR Life Science, Solon, OH) in PBS containing 4 μM menadione (Sigma Aldrich) was introduced into the biofilms. The plates were incubated in dark for 20 min at 37° C. and subsequent colorimetric changes in the XTT reagent was measured at 490 nm using a spectrophotometer (Synergy H1, BioTek).

Sedimentation Assays. S. mutans UA159 cultures grown in UFTYE medium plus 1% (w/v) glucose was harvested by centrifugation at 3900 rpm for 5 min at 4° C. and the cell pellets were washed in PUM (Phosphate Urea Magnesium) buffer. The pellets were suspended in PUM buffer, pH 7.1 to OD ˜1.0 at 600 nm. Additionally, sodium phosphate buffer (20 mM) was also utilized for the sedimentation assay with addition of 0.8 mM MgSO₄ when noted. The cell suspension was transferred into cuvettes (BRANDTECH Scientific) and was exposed to CeO₂—NP, CeO₂—NP-CSA, Ce(NO₃)·6H₂O (250 μM, Ce) and CSA estimated to be present in equivalent amounts of CeO₂—NP-CSA. Cell sedimentation was measured in terms of optical density of the cell suspension at 600 nm every 30 min for up to 2 h based upon a modified literature protocol. ³¹ To determine the effect of mixing on cellular sedimentation, all Ce-containing species were pre-incubated/mixed in PUM buffer, pH 7.1 for 10 min at 37° C. prior to assay initiation (addition of cells).

Microscopy of Planktonic Cell Clusters. S. mutans UA159 cultures were grown to the mid-exponential phase (OD₆₀₀=0.5) in UFTYE medium plus 1% (w/v) glucose. The bacterial culture was exposed to 250 μM (Ce) concentration of CeO₂—NP-CSA after 2 h of incubation in 37° C. with inclusion of 5% CO₂, 5 μL of the cell suspension was spread on a microscope glass slide (on a defined surface area of 2.25 cm²), air dried and stained with 0.1% (w/v) crystal violet. Cellular clustering was visualized under the oil immersion objective of a light microscope (Laxco™) ImageJ software was used to determine the size of the cellular clusters and was calculated using the particle size analysis function for three images from each group and averaged accordingly.

Cell Viability of Clustered Bacteria and Biofilms. Overnight cultures were washed and suspended in PUM buffer and was incubated for 2 hours at 37° C. with inclusion of 5% CO₂ and with and without the supplementation of 250 μM (Ce) CeO₂—NP-CSA. The cell clusters were sonicated for 10 seconds at 20% Amplitude (Qsonica), vortexed and then plated out in BHI agar. The plates were incubated for 48 h at 37° C. with inclusion of 5% CO₂ and the resulting colony forming units (CFUs) were counted. To evaluate the viability of the cells in the biofilm, S. mutans cells were grown in UFTYE+1% wt. sucrose medium treated with CeO₂—NP-CSA (500 μM, Ce) in a μ-slide 8 well plate (ibidi, Germany) for 24 hours in a 5% CO₂ atmosphere at 37° C. Biofilms were rinsed in PBS and stained with a LIVE/DEADM BacLight™ bacterial viability kit (Molecular Probes, OR; excitation: emission—SYTO™ 9: 485/498, Pl: 535/617) following manufacturer instructions and images acquired using an Olympus X70 confocal microscope was visualized using the Imaris X64 9.3.0 software.

Insoluble Glucan Measurement. GIucosyltransferase enzymes (Gtfs) were isolated using a previously published protocol. For the optical density (OD₆₀₀) assay, isolated Gtfs were incubated for 24 h with 1.8% wt. sucrose with and without the CeO₂—NP-CSA (500 μM Ce). Following incubation, the samples were treated with 10 μL of 0.1 M EDTA, mixed (10 min) and incubated to disrupt any existing glucan-CeO₂—NP-CSA surface interactions then the final OD₆₀₀ was measured. In the fluorescent based assay, glucan formation by Gtfs was quantified using Alexa Fluor 647-labeled dextran conjugate (10,000 molecular weight; absorbance/fluorescence emission maxima, 647/668 nm; Molecular Probes). Isolated Gtfs were incubated using similar conditions above, with addition of Alexa Fluor 647 conjugate (9.6 μg/mL final concentration). After 24 h, insoluble glucans (Alexa Fluor 647 conjugate incorporated) produced were centrifuged (10,000 rpm, 5 min), washed twice, resuspended in 200 μL PBS and the fluorescence was measured at 647 nm using a spectrophotometer (Synergy H1, BioTek).

RNA isolation, Library Preparation, Sequencing and Analysis. Sucrose supplemented S. mutans biofilms were exposed to sterile Milli-Q® water (control group) or CeO₂—NP-CSA (250 μM, Ce) for 24 hours. Total RNA was purified from biofilms using an automated Maxwell RSC SimplyRNA Cells kit (Promega) with the inclusion of a DNase treatment step. An additional DNAse treatment was performed to decrease the remaining DNA amounts, followed by purification using RNA Clean & Concentrator Magbead purification kit (Zymo Research). RNA samples were quantified for RNA and DNA content using a Qubit fluorometer (Invitrogen) and analyzed for integrity using Agilent 4200 TapeStation. Levels of remaining DNA did not exceed 10% of the total amount of nucleic acid. Sequencing libraries for Illumina sequencing were prepared in one batch in 96-well plate using CORALL Total RNA-seq Library Prep Kit with Unique Dual Indices (Lexogen, PN M11696) with RiboCop Probe Mix G+ rRNA Depletion Kit (Lexogen PN M12424). In brief, approximately 10 nanograms of total RNA per sample were used for the first rRNA depletion step, then followed by library generation initiated with random oligonucleotide primer hybridization and reverse transcription. Next, the 3′ ends of first-strand cDNA fragments were ligated with a linker containing Illumina-compatible P5 sequences and Unique Molecular Identifiers (UMIs). During the following steps of second strand cDNA synthesis and ds cDNA amplification, i7 and i5 indices as well as complete adapter sequences required for cluster generation were added. The number of PCR amplification cycles was 15 as determined by qPCR using a small pre-amplification library aliquot for each individual sample. Final amplified libraries were purified, quantified, and average fragment sizes confirmed to be approximately 340 bp by gel electrophoresis using 4200 TapeStation and D5000 Screen Tape (Agilent, PN 5067-5588). The concentration of the final library pool was confirmed by qPCR and the pool was subjected to test sequencing on MiniSeq instrument (Illumina) in order to check sequencing efficiencies and adjust accordingly proportions of individual libraries. Sequencing was carried out on NovaSeq 6000 (Illumina), S4 flowcell, 2/150 bp, 60 paired-end reads per sample. It was done at the Roy J. Carver Biotechnology Center at the University of Illinois at Urbana-Champaign. Raw reads were aligned with the reference genome for Streptococcus mutans strain NCTC10449 (NZ_LS483349.1) using BWA MEM. Gene expression was quantified using FeatureCounts. Normalized and differential gene expression statistics were computed using edgeR. P-values were adjusted for multiple testing using the false discovery rate (FDR) correction of Benjamini and Hochberg. An additional log₂ fold cutoff=±0.50 for expression was applied to identify the genes that were most affected.

Human Cell Proliferation Assays. Human telomerase immortalized gingival keratinocytes (TIGK, ATCC, Manassas, VA) were seeded (3000 per well) in a 96-well plate in a culture medium (DermaLife K Medium Complete Kit, Lifeline Cell Technology, Frederick, MD) without the use of antibiotics. 20 mM (metal ion) stock solutions of CeO₂—NP-CSA and NaF filtered through a 0.45 μm filter were used to deliver concentrations ranging from 250-2000 μM in the wells containing the cells and incubated for further 48 h. The amount of CSA estimated to be present in CeO₂—NP-CSA at the above concentrations was treated similarly and added separately to specified wells. Concentrations of CeO₂NP-CSA stock solutions were verified post filtration via UV-Vis Spectroscopy absorbance at 290 nm and the resultant average hydrodynamic diameter (nm) vs intensity spectra (DLS) were collected to verify only minor changes to spectra have occurred from the filtering process. Cells were cultured in an incubator at 37° C. in 5% CO₂ environment. Cells without the addition of any metal ions or CSA was used as the baseline control. Each treatment condition had three replicates. A cell proliferation assay was performed using a colorimetric MTS Assay Kit (Abcam, Waltham, MA) per manufacturer's instructions. The optical density at 490 nm was read using a spectrophotometer (SPECTRAmax Plus, Molecular Devices, San Jose, CA). A similar human cell proliferation assay utilizing the same metal containing solutions was carried out with primary human gingival fibroblasts (HGF, ATCC) cultured in DMEM (ThermoFisher Scientific, Waltham, MA) with 10% fetal bovine serum.

### Data Analysis

XRD data was plotted on Origin (OriginLab Corporation, North Hampton, MA). EELS data was graphed via Kaleidagraph (Synergy Software Inc., Reading, PA). All DLS curves and human and bacteria cell based assays were plotted on GraphPad 9.0 (GraphPad Software Inc., San Diego, CA). For pairwise comparisons, the t test (two way) analysis of all test substances were performed using GraphPad Prism software where p values <0.05 were considered statistically significant, where indicated.

### Results and Discussion

The synthetic preparation of CeO₂—NP-CSA and the advantage of using CSA to promote CeO₂—NP aggregation in acidic media over the use of basic conditions alone is provided in FIG. 1.

X-ray diffraction (XRD) was used to estimate the crystalline size of the initial precipitates (P-1, P-2) prepared from FIG. 1 based on the Debye-Scherrer equation. The average particle size in P-1 and P-2 was calculated based on the three most intense XRD peaks of (111), (220), and (311). Considering the full width half maximum (FWHM) of each of these peaks, the particle size of each sample was obtained from the average of the calculated values from these three peaks. The calculated average particle sizes of the bulk phase precipitate with 1 M NaOH (P-1) is 2.57 nm and the precipitate generated from addition of CSA (P-2) is 2.55 nm. Although these values are lower than those obtained from previous STEM/EELS measurements (3-5 nm), they approach 3 nm when errors in calculations associated with the measurement of small particles including instrumentation broadening, inhomogeneous strain and crystal lattice imperfections, are taken into account. As the calculations based on the Debye-Scherrer equation depends on the diffraction peak broadening, there are many other sources for that, such as dislocations, stacking faults, twinning, micro stresses, grain boundaries, sub-boundaries, coherency strain, chemical heterogeneities, and crystallite smallness. Moreover, this analysis does not take into account the re-dispersion process in NaHCO_{3(aq)} discussed in the next section.

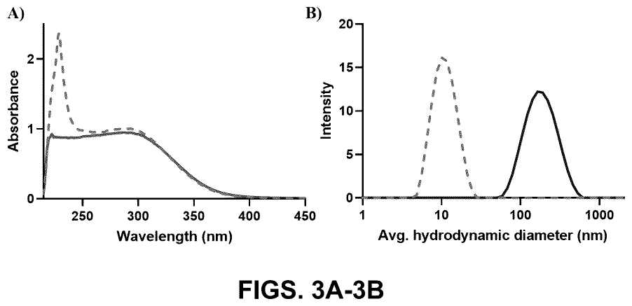

In a routine preparation, P-2 (non-dried) was dispersed in pH 9.5 adjusted NaHCO₃ (30 mM) and buffer exchanged to an estimated concentration of ≈30 mM (Ce) CeO₂—NP-CSA in Milli-Q® water based upon UV-Vis analysis (FIG. 3A) and confirmed by ICP-OES (Ce) analysis. The Blyscan assay²³⁻²⁴ was used to estimate the CSA content in CeO₂—NP-CSA, found to be ≈2.0 mg/mL at 30 mM Ce, also consistent with ICP-OES (S) analysis based on the sulfur content in free CSA. It is important to note that total CSA present in CeO₂—NP-CSA may include small amounts of non-surface adsorbed CSA, following re-dispersion in NaHCO₃(aq) and buffer exchange purification. In a separate experiment, both P-1 and P-2 were dried for four days, stored under reduced pressure and analyzed via FT-IR. P-2 showed evidence of absorption peaks consistent with the presence of CSA, but due to the overlapping region of peaks associated with the Ce—O moiety, no definitive shifts in peak's associated with surface adsorbed —COO⁻¹ and —OSO₃⁻¹ groups could be assigned.

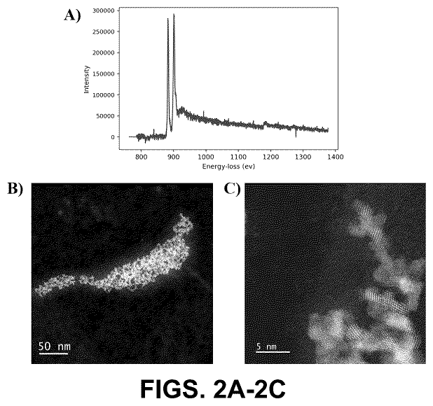

Atomic-resolution imaging and spectroscopy analysis of CeO₂—NP-CSA was performed in an aberration-corrected scanning transmission electron microscope (STEM). Using low-angle annular dark field imaging and electron-energy loss spectroscopy (EELS) of samples suspended in graphene liquid cells, the NP size, distribution and the valence state can be directly studied (FIG. 2A). EELS data was collected from CeO₂—NP-CSA dispersed in water, and found to have a predominant valence state of Ce(IV), similar to literature reports and our previous findings for non-formulated CeO₂—NP (3-5 nm) in water formed from direct ceric salt hydrolysis. Several references have indicated that below a critical NP size (i.e., less than 3-5 nm in diameter) the CeO₂₋ₓ NP primarily consist of Ce(III). In particular, this has been demonstrated for bare nano-particle surfaces in a reducing environment as the result of oxygen loss. The reduced CeO₂₋ₓ NP also show a noticeable increase in lattice parameter that was not observed in the CeO₂—NP-CSA dispersed in water. Scanning transmission electron microscopy (STEM) (FIG. 2B-C) imaging using low-angle annular dark field imaging mode shows individual CeO₂—NP's are highly crystalline and have nearly mono-disperse particle size distributions of 3-5 nm in different crystal orientations. Lower magnification images showed CeO₂—NP-CSA exists as larger aggregates ranging in size from 20 nm to 200 nm. However, these estimates on aggregate sizes are limited by relatively low statistics and the high irregular shape of the aggregates.

In general, UV-Vis Spectroscopy (absorbance 290 nm) of dispersed CeO₂—NP-CSA and non-formulated CeO₂—NP (3-5 nm) was used to estimate the CeO₂—NP concentration (indirectly, the Ce content) from these stock solutions (FIG. 3A). Dynamic light scattering (DLS) showed a large increase in the size of the average hydrodynamic diameter in CeO₂—NP-CSA (≈165 nm) as compared to non-formulated CeO₂—NP (≈9-10 nm) under similar conditions (FIG. 3B).

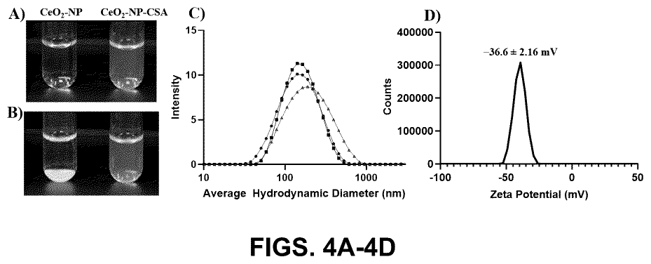

CeO₂—NP-CSA resists bulk precipitation under low ionic strength, pH neutral conditions (pH 7.3-7.8) at 4° C. for several months and also for over 24 h in the presence of two equivalents of sodium fluoride (NaF) (FIG. 4A-B). Prior to activity studies, only minimal vortexing under the above conditions is required to disperse the system. Further, CeO₂—NP-CSA maintains a similar size (i.e., hydrodynamic diameter) over a large pH range (pH 3.0-10.5 in buffered media) (FIG. 4C). The charge brought about by the surface adsorbed CSA (and surface adsorbed ions of the purification process, buffered media) results in a negative ZP in low ionic strength media (−36.6 mV at pH 6.0, 20 mM NaH₂PO₄) (FIG. 4D). The negative ZP helps reduce sedimentation under pH neutral conditions, also in low ionic strength media.

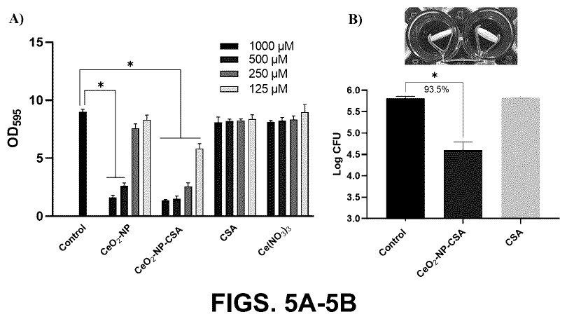

In vitro, static biofilm inhibition assays established the efficacy of the CeO₂—NP-CSA in limiting sucrose-dependent S. mutans adherent biofilm formation on both PS plates and vertically suspended HA discs (FIGS. 5A-5B) in UFTYE medium. Both non-formulated CeO₂—NP (3-5 nm) and Ce(NO₃)·6H₂O were included in the dose-dependent (broth dilution method) PS inhibition studies as both are reported to exhibit anti-biofilm activity under various conditions. CeO₂—NP-CSA was the only test agent to exhibit a statistically significant reduction of adherent biofilm at lower concentrations [250, 125 μM (Ce), p<0.05] under these conditions (FIG. 5A). Ce(NO₃)₃·6H₂O showed no statistically significant reduction in the microbroth dilution method. In the HA disc model, CeO₂—NP-CSA showed a 93.5% reduction (compared to control) in adherent biofilm formation at 250 μM (Ce) (p<0.05; FIG. 5B). Free CSA showed no inhibitory activity on either PS or on the HA discs at the same concentrations present in CeO₂—NP-CSA. A rinse model biofilm dispersion assay of S. mutans UA159 biofilms following a 2 h treatment with 2 mM (Ce) CeO₂—NP-CSA showed no dispersal of sucrose dependent biofilms on a PS surface (24 h growth). In summary, CeO₂—NP-CSA inhibits the initiation or early stages of in vitro biofilm formation to significantly reduce adherent biofilm of S. mutans.

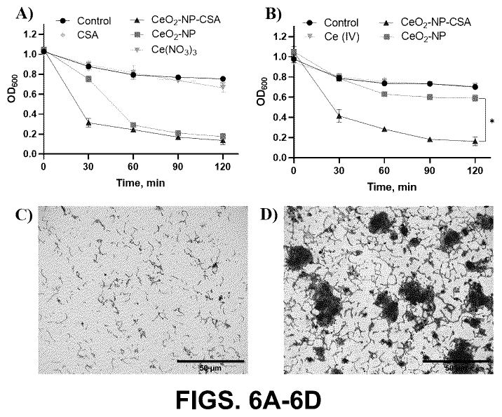

In vitro sedimentation assay was utilized to compare the clustering of S. mutans cells in response to equimolar (Ce) concentrations of CeO₂—NP-CSA, non-formulated CeO₂—NP (3-5 nm) and Ce(NO₃)·6H₂O under buffered conditions. As shown in FIG. 6A, direct addition of test agents to suspended S. mutans cells in PUM buffer [phosphate urea magnesium (PUM) pH 7.1] resulted in a similar clustering of cells between non-formulated CeO₂—NP (3-5 nm) and CeO₂—NP-CSA with no significant activity observed for Ce(NO₃)·6H₂O or free CSA. When a similar assay was carried out by first incubating/mixing CeO₂—NP (3-5 nm) and CeO₂—NP-CSA for 10 min in PUM at 37° C., significant rapid sedimentation of cells was found only for CeO₂—NP-CSA (FIG. 6B). The trend in clustering activity in this pre-mixing assay mirrors the trend in biofilm inhibition observed in the broth (UFTYE) dilution assay shown in FIG. 5A. We hypothesized that CeO₂—NP-CSA better resists decomposition pathways that limit its ability to induce cluster formation. Maintaining clustering efficacy under a variety of conditions is an important feature for both future formulation design as well as intraoral use. The direct addition of equimolar 1 N H₂Ce(NO₃)₆[Ce(IV) ions] to both PUM buffer (neutral pH) and UFTYE resulted in immediate precipitation with no observed clustering of S. mutans or reduction of adherent biofilm, respectively. This suggests that formulating a strong Lewis acid such as Ce(IV) is key to leveraging clustering activity under various buffer or cell growth conditions. S. mutans cell clusters induced by CeO₂—NP-CSA in PUM buffer were viable following treatment at 250 μM (Ce) supporting a non-bactericidal mechanism of action of CeO₂—NP-CSA under similar conditions. Live/dead staining complemented the viability of cells in the retained biofilm after treatment with CeO₂—NP-CSA (500 μM, Ce). Light microscopy of planktonic S. mutans in growth media (UFTYE+1% glucose, 2 h of growth exposed to either water or CeO₂—NP-CSA) (FIG. 6C-D) provided further evidence of clustering as larger cellular clusters were found for CeO₂—NP-CSA treated cells (average size of 69.14 μm²) compared to the control (water) with an average cluster size of 12.08 μm².

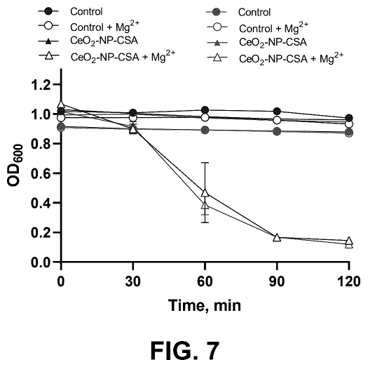

Sedimentation of S. mutans cells in a low ionic-strength buffer (i.e., 20 mM Na₂HPO₄) (FIG. 7) was evaluated as a comparison to a high ionic-strength buffer (PUM). No clustering of S. mutans was immediately observed in the sedimentation assay when treated with CeO₂—NP-CSA (at 250 μM Ce) under low ionic strength conditions. However, the addition of 0.8 mM Mg²⁺ (MgSO₄) resulted in sedimentation of cells in media containing CeO₂—NP-CSA. Although Mg²⁺ enhances the rate of sedimentation by CeO₂—NP-CSA in 20 mM Na₂HPO₄, it is not required for cell clustering in high ionic strength media, as a rapid rate of clustering of S. mutans is observed in PBS buffer devoid of divalent metal ions. DLS data collected on CeO₂—NP-CSA in PUM, PBS and 20 mM Na₂HPO₄ (with 0.8 mM Mg²⁺) is non-determinable with a high polydispersity index (PDI) (data not shown). We propose agglomeration (and/or aggregation) of CeO₂—NP-CSA takes place under high ionic strength conditions (and in the presence of Mg²⁺ in 20 mM Na₂HPO₄), as observed with other nanoceria.

Other non-bactericidal mechanisms accounting for the reduction of adherent S. mutans biofilm by CeO₂—NP-CSA were also investigated. Glucosyltransferase enzymes (Gtfs) utilized by S. mutans are integral to biofilm formation as they produce an array of glucans critical to biofilm adherence. The inhibition of Gtf's is an effective way to reduce adherent biofilm of S. mutans and a potential contributor to the observed reduction in adherent biofilm observed in FIG. 5A-5B. S. mutans synthesize soluble (Gtf-S, GtfD), partially soluble (Gtf-SI, GtfC) and insoluble glucans (Gtf-1, GtfB) in the presence of sucrose. Following modifications to a known literature procedure, Gtfs (GtfB, -C, -D) were identified in the supernatant of UFTYE of which S. mutans was grown overnight. The Gtf containing supernatant was treated with 1.8% sucrose and the resulting insoluble glucans production in the presence of CeO₂—NP-CSA was measured via optical density (OD₆₀₀). Similarly, glucan production was evaluated using a fluorescent based assay where an Alexa Fluor 647 dextran conjugate was added to the reaction mixture (containing 1.8% sucrose, Gtfs and with/without CeO₂—NP-CSA) which labels the produced insoluble glucans, and analyzed using fluorometry. CeO₂—NP-CSA treated UFTYE, in both assays containing GtfB, -C, -D demonstrated no statistically significant reduction of insoluble glucans. The same CeO₂—NP-CSA concentration (500 μM Ce) utilized in the static biofilm inhibition data (FIG. 5A) resulted in >70% inhibition of adherent biofilm. Irreversible Gtf inhibition by CeO₂—NP-CSA at lower concentrations was ruled out as a significant contribution to reduced biofilm adherence, however, potential glucan-CeO₂—NP-CSA interactions that may affect biofilm formation are still under investigation. RNA sequencing analysis of S. mutans treated with CeO₂—NP-CSA grown in the presence of sucrose (24 h) was compared to a control (cells treated with water). In general, few genes shown previously to be critical for S. mutans biofilm formation were significantly affected. The expression of glucosyltransferase genes were affected as follows: gtfD downregulated, while gtfB and gtfC was upregulated with less fold increase. The gene (brpA) encoding the cell surface biofilm regulatory protein A (BrpA), involved in biofilm formation, cell division, autolysis and the regulation of acid and oxidative stress tolerance was significantly upregulated. Further, the genes related to iron metabolism were highly upregulated and the expression of additional genes related to the regulation of oxidative stress tolerance was also significantly altered. As such, it is not possible to rule out contributions from any of the above changes in gene expression (i.e., particularly oxidative stress) that may be linked to cellular clustering or mechanisms of reducing adherent biofilm observed in other studies.

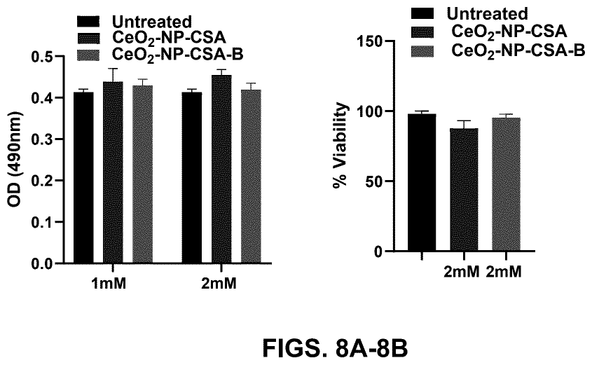

FIG. 8A shows the MTS proliferation assay (OD 490 nm) of seeded HGF cells following 48 h exposure to nanohybrid aggregates at two concentrations. FIG. 8B shows a viability assay (trypan blue dye) was utilized to quantify viability of seeded TIGK cells following 24 h exposure to the listed agents. Readings were taken in both assays FIG. 8A-B 1 h post exposure after washing cells to remove excess nanohybrid aggregates.

In summary, CeO₂—NP-CSA exists as an anionic aggregate structure in low ionic strength buffers and exhibits similar biological activity as the non-formulated nanoceria (3-5 nm) but with improved storage properties at neutral pH (and in biological buffers) allowing further mechanistic studies. CeO₂—NP-CSA reduces in vitro biofilm formation (i.e., PS, HA discs) of S. mutans under dose-dependent (broth dilution method) conditions via the induction of non-adherent cell clusters reducing the initial attachment phase of biofilm formation. Although CeO₂—NP-CSA was non-cidal under the conditions tested, it is a source of high oxidative stress to S. mutans that may contribute to the observed reduction in adherent biofilm and/or clustering process. It is important to note that CSA alone was inactive in both induced cell clustering and biofilm inhibition assays with S. mutans, while non-formulated CeO₂—NP (3-5 nm) has shown efficacy in both assays. Rapidly induced cell clustering by CeO₂—NP-CSA may involve several independent mechanisms that must also take into account the ability of the cells (and potentially CeO₂·NP-CSA) to aggregate as function of the media chemistry. Although induced cell clustering via the disruption of the bacterial cell electrical double layer is a potential explanation, a study of CeO₂—NP-CSA surface charge, aggregation state and interaction(s) with cell surface biomacromolecules (or functional groups) in biologically relevant media should proceed this conclusion. Alternatively, the ability of nanoceria to induce oxidative or hydrolytic cellular damage may also contribute to the rapid cell clustering process observed in this study. In summary, studies involving the interaction of CeO₂—NP-CSA with the S. mutans cell surface (and associated biomacromolecules) that lead to rapid cell clustering are underway, but outside the scope of this study that sets forth the synthetic preparation, chemical characterization, biological activity, initial mechanistic insight and cellular toxicity of CeO₂—NP-CSA.

### Example 2

### Experimental Section

### Materials

All chemical reagents were purchased commercially and utilized without further purification. Ultrapure Milli-Q® water was used to prepare all samples for synthetic preparation, Dynamic Light Scattering (DLS) and Zeta Potential (ZP) measurements, Scanning Transmission Electron Microscopy and Electron Energy Loss Spectroscopy (STEM/EELS) and UV-Vis Spectroscopy. Ceric nitrate (1.0 N aqueous solution) was purchased from GFS Chemicals (Columbus, Ohio). Chondroitin sulfate A, sodium salt (90%) was purchased from Alfa Aesar (Ward Hill, MA). Dextrose, sucrose, NaHCO₃, NaOH were all purchased from Fisher Scientific (Fair Lawn, New Jersey). Disposable BrandTech PMMA cuvettes (Cole-Parmer, Vernon Hills, IL) and folded capillary cells (Malvern Instruments, Worcestershire, UK) were used for size and zeta potential measurements. Streptococcus mutans (S. mutans) UA159 was prepared from frozen glycerol stock and was maintained in brain heart infusion (BHI; Hardy Diagnostics, Santa Maria, CA). PerioMed™ (3M) was purchased from Amazon and diluted as directed prior to administration. Solid media were prepared by adding 1.5% (w/v) Bacto agar (IBI Scientific, Peosta, IA). Unless otherwise stated, cultures were maintained in an aerobic chamber at 37° C. with inclusion of 5% CO₂. pH measurements were carried out on a benchtop Fisherbrand Accumet AB200 pH (Fisher Scientific, Singapore) with either a i) Fisherbrand Accument liquid-filled mercury-free pH/ATC epoxy body combination electrode (Fisher Scientific, Singapore) for buffered media measurements or ii) an Orion ROSS Sure-Flow Combination pH electrode (Thermo Scientific, Singapore) for solutions containing whole human saliva (WHS). WHS filtration was performed using 0.22 μm Sartorius Minisart™ high flow syringe filters (Sartorius Stedim Biotech Gmbh, Gottingen, Germany).

### Preparation of Nanohybrid Aggregate Formulations

CeO₂—NP (3-5 nm). The synthesis and characterization of CeO₂—NP was carried out as provided in Example 1. A 10 mM stock dispersion was stored at 4° C. prior to use.

CeO₂—NP-CSA. The synthesis and characterization of CeO₂—NP-CSA was carried out as reported previously. ¹⁷ A 30 mM stock dispersion was stored at 4° C. prior to use.

CeO₂—NP-CSA-B. A 50 mL, 10 mM dispersion of non-formulated CeO₂—NP (3-5 nm) was prepared from the hydrolysis of 1 N H₂[Ce(NO₃)₆] and allowed to stand greater than 24 h at room temperature. To this dispersion, 500 μL of a 10% (wt.) solution of chondroitin sulfate A, sodium salt (Acros) was added causing formation of a cloudy off-white precipitate. This mixture was vortexed for 1-2 minutes and allowed to stand at room temperature for approximately 5 min. The resultant mixture was centrifuged for 1 min (3900 rpm, 4° C.). The supernatant was decanted from the precipitate, and 12-15 mL of Milli-Q® water was added to the precipitate, re-dispersed with gentle vortexing and the supernatant decanted and this cycle was repeated (5×). Following removal of the supernatant from the final wash, a fresh 30 mM NaHCO₃ (≈12 mL) solution adjusted to pH 9.5 (with 5 M NaOH) was added to the off-white precipitate, gently vortexed and allowed to stand overnight at room temperature at which time continued aggregation occurs. The next day the concentration was adjusted utilizing fresh 30 mM NaHCO₃ buffer (pH 9.5) to arrive at a concentration of ≈30 mM (Ce) as estimated from UV-Vis Spectroscopy (Absorption 290 nm). ICP-OES (Ce) analysis confirmed Ce content in CeO₂—NP-CSA-B with a total yield of ≈85% (recovery of Ce). At a concentration of 30 mM (Ce) CeO₂—NP-CSA, the CSA concentration is estimated≈2.8 mg/mL from both the Blyscan assay and S-ICP analysis, which is a ≥25% increase in CSA content per Ce as compared to CeO₂—NP-CSA. 17 CeO₂—NP-CSA-B was stored at 4° C. for >6 months with little change to the Z-average diameter (Z-Avg) polydispersity index (PDI) or zeta potential (ZP). The pH of each CeO₂—NP-CSA-B dispersion was approximately 8.3-8.7. Only minimal vortexing was necessary to disperse the aggregates prior to use even after 6 months of storage. Replicate stock preparations of CeO₂—NP-CSA-B (30 mM) were utilized throughout this study to average out small differences in aggregate sizes or CSA content between replicate batches.

### Characterization of CeO₂—NP-CSA-B.

Inductively Coupled Plasma Optical Emission Spectroscopy (ICP-OES). ICP-OES sample analysis was carried out at the University of Illinois Urbana-Champaign (UIUC) Microanalysis Laboratories (Champaign, Illinois). Ce-analysis was carried out by first estimating the concentration by the UV-Vis absorbance at 290 nm and then carrying out a series of dilutions in Milli-Q® water to an estimated concentration of 140 ppm (1 mM Ce). All standard samples (e.g., CeO₂—NP) and test agents (CeO₂—NP-CSA-B, PerioMed™) were digested with a mixture of 2.5 mL hydrogen peroxide (30%), 5 mL sulfuric acid (97%), and 2.5 mL Milli-Q® water for cerium analysis and 5 mL nitric acid (70%), 2 mL of hydrochloric acid (36%), and 2 mL of hydrogen peroxide (30%) for sulfur analysis and Sn analysis. The sample solutions were then subjected to the CEM MARS6 microwave digestion system utilizing the following parameters: Power 700-1800 W, Temperature 240° C., Ramp Time 30 min, Hold Time 15 min. Following complete digestion, the samples were diluted to a 25 mL final volume. Standard calibration curves for both Ce and S were prepared in 2% nitric acid. Samples and standards were analyzed on a Perkin Elmer Optima 8300 ICP-OES instrument with the following parameters: Plasma (Argon) gas flow: 10 L/min; Auxiliary gas flow: 0.20 L/min; Nebulizer gas flow 0.8 L/min; RF Power 1500 W and a pump flow 1.5 mL/min. The emission wavelengths were used at 418.66 nm for Ce, 181.975 nm for S, and 189.927 nm for Sn using axial view. Ce and S concentrations were calculated via linear regression (R²>0.999). A QC standard was run after the samples, which was within ±10% of the expected concentration.

Blyscan Assay. The Blyscan assay (Biocolor Ltd, Antrim, UK) was carried out to determine the concentration of CSA in the formulation of CeO₂—NP-CSA-B. Following serial dilutions of the unknown samples, CeO₂—NP-CSA-B, the concentration of CSA was estimated from the best fit standard curve (linear least squares fit) plot. Each new batch of CeO₂—NP-CSA-B prepared was analyzed via the Blyscan assay for CSA content.

High Resolution Scanning Transmission Electron Microscopy (STEM) and Electron Energy Loss Spectroscopy (EELS). CeO₂—NP-CSA-B was diluted to 5 mM (Ce) in Milli-Q® water and stored at cold temperatures prior to measurements. The atomic-resolution STEM and EELS maps were collected using the JEOL ARM200CF aberration corrected STEM with a cold-field emission gun operated at an acceleration voltage of 200 kV and 80 kV. EELS for sample CeO₂—NP-CSA-B was taken at 80 kV. Low-angle annular dark field (LAADF) imaging was also utilized. The LAADF images were acquired using an annular dark-field detector with a collection angle ranging from 30 to 120 mrad and a probe current of 19 pA. For EELS measurement, the probe convergence semi-angle was set to 17.8 mrad, which yields a probe size of 1 Å and a probe current of 62 pA. EELS characterization was conducted using the post-column Gatan Continuum GIF spectrometer using a collection semi angle of 53.4 mrad. Samples were encapsulated in graphene liquid cells with aqueous media as previously reported. STEM images were recorded via Gatan Digital Micrograph as part of the Gatan Microscopy Suite (GMS, Gatan Inc., Pleasanton, CA) and the EELS spectrum was graphed via Kaleidagraph (Synergy Software Inc., Reading, PA).

UV-Vis Spectroscopy. All UV-Vis spectra were obtained utilizing a NanoDrop Onec Spectrophotometer (Thermo Scientific). Absorbance of dispersions of CeO₂—NP-CSA-B (diluted in Milli-Q® water) were used to estimate the concentration of CeO₂—NP (290 nm) in disposable UV-Cuvettes (BrandTech). Quantification of sedimentation assays was measured by absorbance (OD₆₀₀) in various buffered media.

Dynamic Light Scattering (DLS) and Zeta Potential (ZP) Measurements. All size and zeta potential data was collected utilizing a Malvern Zetasizer Nano ZS in the laboratory of Prof. Richard Gemeinhart (UIC). The average hydrodynamic diameter and Z-average diameter (both in nm) of each sample was determined via the function of intensity weighted distribution of the Malvern software program. All samples were dispersed in Milli-Q® water (18.2 MΩ) based buffers (30 mM NaH₂PO₄, 30 mM NaOAc) at 25° C. and measured in either disposable cuvettes or capillary folded disposable capillary cells (Malvern). ZP measurements were made at the time of the size vs intensity experiments described above under similar conditions. All hydrodynamic (or Z-Avg) diameter and ZP measurements were carried out with a minimum of three and five separate runs, respectively, and reported as an average and standard deviation of these replicate measurements. The long-term storage stability study was carried out using the above protocols (in 30 mM NaH₂PO₄) in the analysis of the same sample of CeO₂—NP-CSA-B stored at 4° C. over the course of >6 months.

### Whole Human Saliva (WHS) Studies

WHS Collection and Processing. Unstimulated WHS was collected by the passive drool method from up to 4 healthy (ASA I) adults (M/F), pooled and stored briefly on ice prior to processing. Subjects refrained from consuming food or beverage (except water) for 2 h prior to WHS donation. The pooled WHS samples were processed via two methods for this study. (WHS-1) The pooled samples were centrifuged at 4° C. for 5 min (1950 rpm) to remove particulates. The supernatant was collected and passed through a 0.22 μm Sartorius Minisart™ high flow syringe filter (Sartorius Stedim Biotech Gmbh, Gottingen, Germany) and diluted to a concentration of approximately 50% or 12.5% WHS in Milli-Q® water or specified buffers as dictated by each experiment. (WHS-2) The pooled WHS sample was centrifuged at 4° C., 3900 rpm for 30 min. The supernatant was collected and diluted 50% with Milli-Q water for sedimentation assays and photos. From three separate collections, the average pHs were 7.48 (±0.09) for 100% WHS-1 and 7.37 (±0.17) for 100% WHS-2 and the protein content (Biuret method) was 0.59 (±0.07) mg/mL and 0.74 (±0.08) mg/mL for WHS-1 and WHS-2, respectively. The study was IRB approved and supported by protocol number 2023-0562.

WHS Based DLS Studies. To monitor the aggregation properties of CeO₂—NP (3-5 nm), CeO₂—NP-CSA and CeO₂—NP-CSA-B under physiologic conditions, samples at a concentration of 500 μM (Ce content) were analyzed for size, PDI, and ZP. The samples were incubated in 30 mM sodium acetate buffer (pH 5.0) and 30 mM sodium phosphate buffer (pH 7.0), both with and without 12.5% (v/v) WHS-1, at 37° C. for 60 minutes. At each time point, aliquots were collected and diluted fivefold in the respective buffer before DLS and zeta potential analyses. WHS-1 samples were used within 48 hours to preserve quality. Three independent preparations of each nanohybrid were analyzed in triplicate for each condition.

### Cell-Based Activity Assays

Sedimentation Assays. Assay was performed with slight modification to a previous protocol. S. mutans UA159 cultures grown in ultra-filtered tryptone yeast extract (UFTYE) medium plus 1% (w/v) glucose was harvested by centrifugation at 3900 rpm for 5 min at 4° C. and the cell pellets were washed and resuspended in either buffer described in the DLS/ZP section above, or in WHS-1 (or WHS-2). The optical density of the cell suspensions were adjusted to OD ˜1.0 at 600 nm and transferred into cuvettes (BRANDTECH Scientific) and was treated with CeO₂—NP-CSA-B, CeO₂—NP (3-5 nm) and/or CSA estimated to be present in equivalent amounts of CeO₂—NP-CSA-B. Cell sedimentation was measured in terms of the reduction of OD₆₀₀ as a function of time as measured by UV-Vis analysis.

Static Biofilm Inhibition Assay. WHS-1 was diluted 8-fold (i.e., 12.5%) in UFTYE+1% sucrose medium. Overnight S. mutans UA159 cultures were transferred to UFTYE+1% glucose medium and grown until the mid-exponential phase (OD₆₀₀=0.5) and diluted 1:100 in the above 12.5% WHS-1 containing medium and transferred to a 24 well plate containing hydroxyapatite (HA) discs vertically suspended using orthodontic wires. The wells were treated with test agents at a final concentration of 500 μM. The cells were incubated at 37° C. for 24 hours aerobically in the presence of 5% CO₂. Following incubation, the HA discs were sonicated (20% amplitude for 10 seconds on ice) and vortexed to remove the attached biofilm cells. The cell suspensions were serially diluted in PBS and plated on BHI agar for S. mutans enumeration.

Static Biofilm Dispersal Assay. WHS-1 was coated on HA discs vertically suspended using orthodontic wires in a 24 well plate and by incubating for 1 hour at 37° C. Following incubation, WHS-1 was replaced with mid exponentially (OD₆₀₀=0.5) grown S. mutans UA159 cultures diluted 1:100 in UFTYE+1% sucrose medium and incubated for 24 hours aerobically in the presence of 5% CO₂. The cells and media were removed and the HA discs were treated with test agents at a concentration of 30 mM for 10 seconds. Following a washing step, the HA discs were sonicated and vortexed to detach the biofilm cells, serially diluted and plated on BHI agar for S. mutans enumeration.

Human Cell Toxicity Assay. CeO₂—NP-CSA-B was prepared as a 30 mM stock solution, transferred to 1.5 mL cuvettes, and sterilized under UV light for 1 hour in a tissue culture hood. Following sterilization, stock dispersions were stored at 4° C. for <8 months for use in in vitro assays. To assess whether each nanohybrid aggregate was cytotoxic under specified conditions, we performed a 48 h cytotoxicity assay. Primary human gingival fibroblasts (HGF) were seeded at a density of 1.5×10⁴ and incubated with nanohybrid aggregates for 48 hours. Subsequently, cells were washed once with PBS to remove all treatment reagents. An MTS assay (Abcam) was then used to assess cytotoxicity. Optical density at 490 nm was determined immediately after exposure using a microplate spectrophotometer (Molecular Devices) following one hour of incubation with the MTS reagent.

### Rodent Model Studies

Disease Induced Caries Model (efficacy study): Rodent model studies were carried out with thirty-six, male Sprague-Dawley (SD) rats (Charles River Breeding Laboratories, antibody and virus free). On day 17 (animals age), the pups were screened for the presence of S. mutans via oral swabs plated on Mitis Salivarius Bacitracin (MSB) agar. No animals were found to be S. mutans positive prior to inoculation. On day 21 (age of animals) the rodents were weaned from the mother and inoculated for the first time with S. mutans UA159 grown to log phase (OD₆₀₀=˜0.5) in BHI media. Inoculation was carried out via syringe addition into the buccal cavity of each rodent (0.1 mL total, 0.05 mL each side) on day 21 and 24 (age of animals). The animals were swabbed for the detection of S. mutans one day after the second inoculation confirmed by presence on MSB agar plates. NIH cariogenic diet 2000 (test diet, St. Louis, MO) and 5% sucrose water was given to the rodent's ad libitum beginning at the time of the first inoculation with S. mutans. The rodents were tagged and separated into three groups of 12 rodents. Group 1) water treated, Group 2) PerioMed™ treated (dilutions according to manufacturer's specifications) and Group 3) CeO₂—NP-CSA-B treated. Treatment began 2 days following confirmation of the presence of S. mutans. Treatment began via syringe addition of agent (rinse model) with 0.05 mL on each side of the buccal cavity for a total of 0.1 mL per treatment, once per day in the rodents who were between 50-100 g at the time of initial dosing (1 mL/kg rodent). Rodents were not allowed to eat/consume 5% sucrose water within 2 h of treatment. The treatment continued from days 1-21, where animals were observed 1-2 h post dosing for clinical signs of toxicity. The animals were sedated (ketamine: xylazine, 100 mg/kg: 5 mg/kg) and euthanized via CO₂ asphyxiation followed by cervical dislocation (necropsy) on day 21 of treatment. Scoring was caried out as described below. The study was approved by the UIC animal care committee and supported by protocol number 21-075.

Plaque scoring: Following euthanasia described above, rodent molars were dabbed with Youngs 2tone disclosing solution (blue-violet) and blotted with a dry cotton roll/gauze pad.

Tooth retained plaque was scored following literature using the Park and Katz methods. In this method, teeth were given a score from 0 to 3, in which 0=no dental plaque detected; 1=dental plaque covering less than one third of the gingival half of the tooth; 2=dental plaque covering between a third and two thirds of the gingival half of the tooth; and 3=dental plaque covering two thirds or more of the tooth. Plaque scoring was reported by a separate examiner who was trained and blinded.

Visual caries scoring: Following euthanasia, the jaws of each rodent were stored in 10% buffered formalin and de-fleshed (hand stripping) according to literature protocols described by Larson. Caries were scored visually with the aid of magnifying lenses (2-4× magnification) and LED light generally following the visual criteria outlined in Larson's modification of Keyes method ²⁶ by a single examiner (pediatric dentist) who was blinded in reference to each treatment group. Caries were recorded on each rodent molar (all four quadrants) based on enamel breakthrough and indicated as either caries or no-caries. All surfaces on all 144 teeth of each group were evaluated individually (occlusal, buccal, lingual, mesial, distal). To facilitate better visualization of proximal surface caries, a small forceps was placed proximally between teeth with slight elevation.

### MTD and Toxicity Study

Growth Study: A maximum tolerated dose (MTD) study was carried out with Sprague-Dawley rats (7 weeks age, 150-200 g) separated into 4 groups of three. Group 1 was the control group (water treated), group 2 was the low-dose (1 mL/kg), group 3 was the mid-level dose (5 mL/kg), and group 4 was the high-dose (10 mL/kg) treatment of CeO₂—NP-CSA-B. All animals were given a single dose via oral gavage on day zero and scheduled for necropsy on day 9, post-treatment. Daily body weights were recorded, clinical signs of pathology were noted 1-2 h post dosing and once daily thereafter. No visual signs of pathology (or animal deaths) were found by the end of the 9-day study in any control or treatment group.

Clinical Chemistry and Hematology Analysis: Blood was collected from the orbital sinus of each animal following anesthesia induced by CO₂(g) (70% CO₂/30% O₂) on day 4 following similar dosing concentrations of CeO₂—NP-CSA-B as described above on day 0. Blood samples were processed via standard methods to determine effects on clinical chemistry and hematological parameters.

### Data Analysis.

EELS data was graphed via Kaleidagraph (Synergy Software Inc., Reading, PA). All DLS curves and human and bacteria cell-based assays were plotted on GraphPad 10.0 (GraphPad Software Inc., San Diego, CA). GraphPad Prism software was also used for comparative analysis (ANOVA) in cell-based assays (where noted) where non-treatment vs treatment were analyzed, and statistical significance (p value) was evaluated based on the type of data acquired (i.e., efficacy, toxicity). All rodent data (efficacy, MTD studies) were analyzed via Kruskal-Wallis non-parametric ANOVA with Dunn's post-hoc test, also on GraphPad. The schematics throughout document were created using BioRender.com.

### Results and Discussion

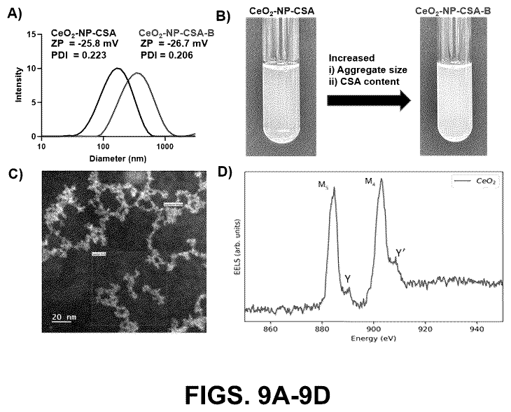

CeO₂—NP-CSA-B was prepared via a large-scale reaction comprising the addition of 10% wt. CSA to 10 mM CeO₂—NP (3-5 nm) to yield an off-white precipitate that was redispersed in 30 mM NaHCO₃. In contrast to the preparation of CeO₂—NP-CSA that was buffer exchanged and stored in Milli-Q® water (pH˜7.5), CeO₂—NP-CSA-B was not buffer exchanged and stored in 30 mM NaHCO₃ (pH 8.3-8.7). Avoiding the buffer exchange step afforded large scale preparation and storage of CeO₂—NP-CSA-B in a buffer utilized in commercial mouth rinses. Under the the same conditions (30 mM NaH₂PO₄, pH 7.0), CeO₂—NP-CSA-B has a similar polydispersity index (PDI) and zeta potential (ZP) as CeO₂—NP-CSA (FIG. 9A). However, CeO₂—NP-CSA-B has a significantly larger hydrodynamic diameter and absorbed CSA content as compared to CeO₂—NP-CSA (FIGS. 9A-B).

Atomic-resolution imaging and spectroscopic analysis of CeO₂—NP-CSA-B was performed in an aberration-corrected scanning transmission electron microscope (STEM), similar to our previously reported methodology. Using low-angle annular dark field imaging (LAADF) (FIG. 9C) and electron-energy loss spectroscopy (EELS) (FIG. 9D) of samples suspended in graphene liquid cells, the NP size, distribution and the valence state can be directly studied. The LAADF image contains monodispersed nanoceria (3-5 nm) as previously published with CeO₂—NP-CSA, existing as aggregates in the estimated size range of 50-300 nm, but a clear statistical difference in aggregate size vs CeO₂—NP-CSA of our previous publication could not be made off these measurements due to shape irregularities. Further the intensity of M₄ peak is greater than that of M₅ peak indicating that the particles are CeO₂—NP, and the nanoceria are predominately Ce(IV). The presence of smaller Y and Y′ peaks also indicate the same.

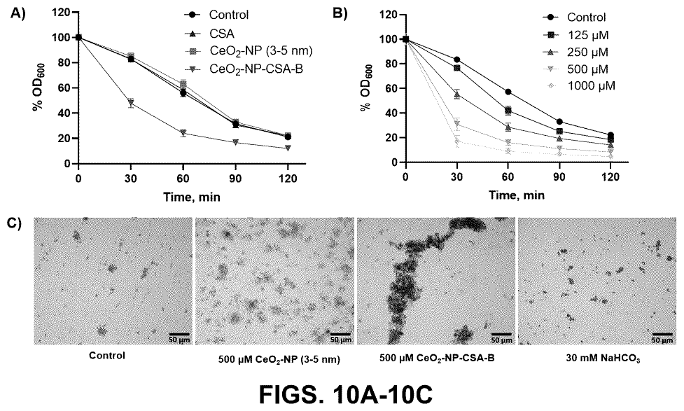

Initially, the goal was to evaluate the efficacy of CeO₂—NP-CSA-B in clearing S. mutans cells from WHS containing samples compared to other Ce-containing agents [CeO₂—NP (3-5 nm), Ce(NO₃)₃]. In 50:50 WHS-1, CeO₂—NP-CSA-B was the only agent to rapidly sediment cells (FIG. 10A). At higher concentrations more likely to be encountered from oral administration, CeO₂—NP-CSA-B showed immediate sedimentation of cells over both CSA alone (no activity) as well as CeO₂—NP (3-5 nm). The addition of CeO₂—NP (3-5 nm) at higher concentrations (1000 μM, Ce) resulted in a cloudy mixture likely from offsite reactivity with biomolecules present in WHS and/or from a significant acidification of the media. CeO₂—NP-CSA-B was also found to clear S. mutans in a dose-dependent manner in both WHS-1 (FIG. 101B) and non-filtered WHS-2, showing efficacy under more clinically relevant conditions and also higher dispersibility (as compared to CeO₂—NP) under the same conditions. It should be noted CeO₂—NP-CSA has similar clearance activity under the above specified conditions (data not shown). Further evidence supporting the formation of induced cell clusters by CeO₂—NP-CSA-B in WHS was found in a series of light microscopy studies under similar conditions. Compared to untreated (water only) and NaHCO₃ treated cells, several large clusters (>50 μm) were only found following the treatment with CeO₂—NP-CSA-B (FIG. 10C).

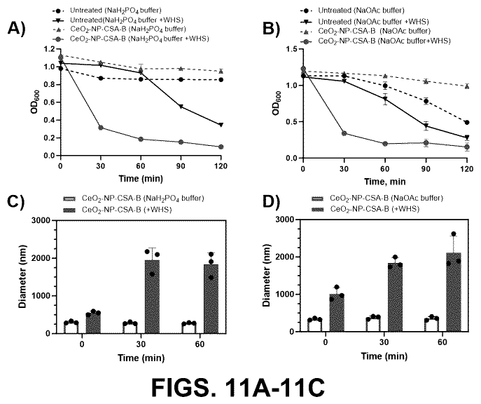

Next, we aimed to correlate the observed sedimentation activity of S. mutans by CeO₂—NP-CSA-B in WHS-1 with parameters collected from time dependent DLS studies. CeO₂—NP-CSA-B remains inactive towards S. mutans in buffer alone [NaH₂PO₄ (pH 7.0); NaOAc (pH 5.0)] similar to reported previously with CeO₂—NP-CSA. However, repeating the sedimentation assay with 12.5% (WHS-1) under identical conditions caused an immediate and rapid sedimentation of cells in the first 30 min in both buffers (FIGS. 11A-B) (similar to CeO₂—NP-CSA). At pH 7.0, without the addition of WHS-1, CeO₂—NP-CSA-B maintained a consistent size over the first 60 min at 37° C. However, the addition of WHS-1 (12.5%) to both buffered solutions resulted in a rapid increase in aggregate size (FIG. 11C-D). Further, a significant increase in PDI and reduction in ZP under the same conditions was noted in both buffers for both nanohybrid aggregates. Whether the rapid increase in sedimentation activity by CeO₂—NP-CSA-B (and CeO₂—NP-CSA) is attributed solely to the increase in aggregate size and/or reduction of ZP (or a combination of both) cannot be determined without further experimentation that is beyond the scope of this study. 12.5% WHS-1 conditions were utilized in these experiments because higher WHS-1 concentrations (25%) introduced significant noise in DLS experiments, likely due to biomolecules interfering with nanohybrid aggregate peaks. Although we cannot prove the high rate and extent of aggregation of both CeO₂—NP-CSA-B (and CeO₂—NP-CSA) in more concentrated WHS containing samples (50%) that may be found in vivo, it should be emphasized that similar rapid and effective clearance of cells occurs under these conditions (FIG. 10A). Although aggregation of MₓO_{y}—NPs (including nanoceria) on bacterial cell surfaces is known under a range of conditions we could find no literature examples of clearance activity of MₓO_{y}—NPs in WHS based systems. This is further underscored by our attempts to carry out analogous time dependent DLS and sedimentation assays with CeO₂—NP (3-5 nm). Due to the limited stability and dispersibility of CeO₂—NP at physiological pH, no reliable DLS parameters could be recovered in WHS based systems in the present study. We hypothesize the anionic CSA coating of the nanohybrid aggregates affords them their unique properties in WHS, including their ability to aggregate in a manner maintaining dispersal under conditions that mimic the oral cavity. Further, it is possible the CSA coating facilitates interaction(s) of the nanohybrid aggregates with the cell surface of S. mutans (and other oral pathogens). However, such conclusions are outside the scope of this study and will be described elsewhere.

Although CeO₂—NP-CSA-B induced sedimentation of cell clusters of S. mutans in media comprising WHS-1, the extent of biofilm inhibition or dispersal in a sucrose-based media required further experimentation. Overnight cultures of S. mutans cells were diluted into media containing UFTYE+1% sucrose and 12.5% WHS-1 and transferred to a 24 well plate containing hydroxyapatite discs (HA) vertically suspended in the media using orthodontic wires. The wells were treated with a series of Ce-containing agents and NaHCO₃ at a final concentration of 500 μM and biofilm formation on the HA disc was evaluated using bacterial cell counting techniques. Only CeO₂—NP-CSA-B was found to significantly inhibit S. mutans biofilm, as compared to equimolar concentrations of cerium nitrate (CeNO₃·6H₂O) or NaHCO₃ (also at 30 mM).

An in vitro human cell toxicity study was carried out with CeO₂—NP-CSA-B against human gingival fibroblasts (HGF). Following 48 hour exposure of HGF to CeO₂—NP-CSA-B, no statistically significant reduction in proliferation (as measured by OD₄₉₀) was found at either 1 mM or 2 mM from the MTS assay.

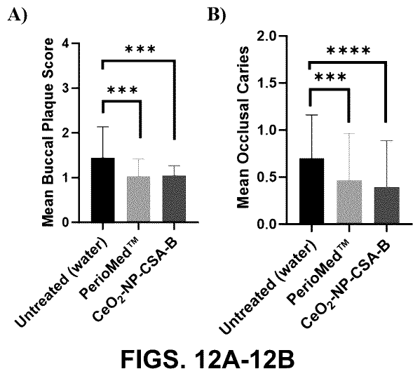

The disease induced caries model of rodents were carried out with modifications to known protocols. The rodent model utilized three groups of 12 rodents (M) inoculated twice with S. mutans at the onset of the study and fed the NIH cariogenic diet and supplemented with 5% sucrose (ad libitum) prior to the initiation of dosing with either water (untreated), PerioMed™ (0.63% wt. SnF₂ or 40 mM Sn) and CeO₂—NP-CSA-B (30 mM Ce). PerioMed™ was chosen as the standard because it contains the highest dose available OTC for fluoride treatment as a rinse application, and it allows for a direct comparison based on bioactive metal ion content in the two formulations (Sn vs Ce). CeO₂—NP (3-5 nm) was not used as a test agent of comparison in the efficacy study due to the inherent acidity found at high concentrations (pH<1.5) and potential widespread damage to the oral tissue of rodents. Three separate sample preparations of CeO₂—NP-CSA-B were prepared and stored at 4° C. and utilized during the caries model study to account for minor variability in sample consistency. Both control and test agents were dosed (via syringe—50 μL) on both upper and lower molars once a day for a total of 100 μL per rodent for 21 days. This is estimated at approximately ˜1-2 mL/kg based on the rodents weight at the beginning of the trial to those at the end. Following the termination of the study and euthanasia of rodents the adherent tooth plaque was scored (FIG. 12A) and the caries were scored visually with the aid of magnification utilizing guidance from Larson's modification of the Keye's method. Molars in all four quadrants (totaling 144 teeth per group) were scored in both the plaque staining and caries analysis, noting that molars exhibiting a minimum of enamel breakthrough on any surface (e.g., occlusal, mesial, distal, buccal, lingual) were recorded as carious by a pediatric dentist blinded from the treatment groups.

Compared to the untreated (water only) group, a similar reduction in both mean plaque score per group and mean caries per molar in each group was found between CeO₂—NP-CSA-B and PerioMed™. The dominant contributor to total caries across all groups was the occlusal surface (FIG. 12B). It is important to note that the plaque scoring and caries analysis were carried out by different examiners, both blinded from the treatment groups. Further, equivalent efficacy of CeO₂—NP-CSA-B to PerioMed™ in both plaque and caries reduction was achieved with 25% less metal ion (Ce vs Sn) and no fluoride content. Mechanistic evidence in static models supports that agglutination (clearance) is a key contributor to the in vivo efficacy of CeO₂—NP-CSA-B against S. mutans, although other potential mechanisms may contribute to a reduction in adherent biofilm, especially in vivo. A 30 mM CeO₂—NP-CSA-B dispersion contains an equal molar amount of NaHCO₃ (0.25% wt) that is often utilized in commercial mouth rinses as a buffering agent, albeit at significantly higher concentrations. Neither in vitro S. mutans sedimentation nor biofilm inhibition assays utilizing solely 30 mM NaHCO₃ produced significant results similar to CeO₂—NP-CSA-B. While others have reported caries reduction in rodent models via dosing with a self-aggregating peptide 33, to the best of our knowledge, this represents the first example of a nanohybrid aggregate formulation (comprising CeO₂—NP and CSA) to achieve caries prevention in rodent models. In addition, the preparation and storage of CeO₂—NP-CSA-B is both facile and cost effective for widespread application in future studies.

In another experiment, periodontal pathogens were grown in respective media in the presence of CeO₂—NP-CSA or CeO₂—NP-CSA-B. Aggregatibacter actinomycetemcomitans (Aa), Porphyromonas gingivalis strain ATCC 33277, Prevotella intermedia (Pi). Inhibition was expressed as % Reduction compared to the non-treatment control. At 1 mM, both formulations inhibited growth/biofilm to varying extent. P. gingivalis 33277 was most susceptible to both formulations. The results are summarized in the table below.

| | % Reduction | | |
|---|---|---|---|
| | Pg | Pi | Aa |
| CeO₂-NP-CSA | 73.72 | 16.54 | 22.51 |
| CeO₂-NP-CSA-B | 71.00 | 57.86 | 32.65 |

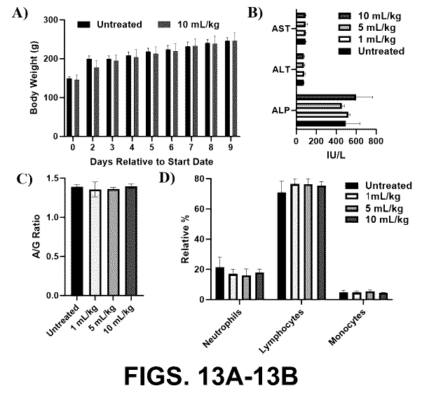

New therapeutic agents require extensive safety data prior to the initiation of clinical studies. A MTD (maximum tolerated dose) study was carried out in adult (M) Sprague-Dawley rats dosed separately (via gavage) with three separate doses of CeO₂—NP-CSA-B (1, 5, 10 mL/kg of 30 mM CeO₂—NP-CSA-B), up to approximately 4× the dose administered in the efficacy studies above. Initial tests followed the change in body weight over nine days post gavage with no evidence of reduced growth, including at the highest dose tested (FIG. 13A). Serum clinical chemistry studies were carried out to determine changes to key markers of systemic health in rodents in response to dosing.³⁴ No significant serum changes reflective of liver and/or kidney damage were found over the range of CeO₂—NP-CSA-B administered (FIGS. 13B-C). Similarly, common hematological markers showed no significant differences as compared to the untreated samples (FIG. 13D). We hypothesize the lack of toxicity with CeO₂—NP-CSA-B is due to a combination of the natural (non-toxic) CSA coating as well as the anionic aggregate nature of this formulation making crossing biological membranes less facile. However, additional long term toxicity and Adsorption Distribution Metabolism Elimination (ADME) studies are needed to confirm this finding.

### Example 3

### Experimental Section

### Protocol Summary for Antioxidant Assays in SOD and CAT Assay

SOD Assay. In general, the assay was carried out under the guidance of the package insert from the supplier (Sigma) in a 96 well polystyrene plate. All control assays were carried out in 100% SOD dilution buffer supplied in the assay kit and indicated in the package insert. Changes were made when incorporating the use of whole human saliva (WHS) into this assay. 33% WHS-1 (see manuscript reference for preparation of WHS-1, IRB #2023-0562) and 67% Assay buffer. All nanoceria samples were diluted onto the 96 well plate at a concentration of 10 μM Ce as the test agent and compared to a negative and positive control and the SOD enzyme itself. The assay was run at 37° C. for over 60 minutes, with readings taken at 450 nm every two minutes (See Avin Protocol).

CAT Assay (in MCM). In general, the assay was carried out under the guidance of the package insert from the supplier (Invitrogen) in a 96 well polystyrene plate. All control assays were carried out in 100% TRIS buffer supplied in the assay kit and indicated in the package insert. Changes were made when incorporating the use of mucin containing media (MCM, Forsythe Institute Recipe) or Biotene™ (Haleon) into this assay. All assays were carried out in 33% MCM and 67% TRIS (kit buffer, pH 7.0) at 37° C. for over 60 minutes, with readings taken at 560 nm every two minutes (See Avin Protocol).

CAT Assay (in Biotene). The assays were carried out similar to above except in 33% Biotene™ and 67% TRIS (kit buffer, pH 7.0) Nanoceria samples were diluted to 6 mM in 33% Biotene™/67% TRIS and allowed to stand 24 hour at 4 deg C. They then were diluted onto the 96 well plate at a concentration of 150 μM Ce as the test agent and compared to a negative and positive control and the CAT enzyme itself, all in approx. 33% Biotene™:67% TRIS. Samples were run for 120 min at 37 deg and measurements were taken at 560 nm every 5 minutes.

For the Photo (showing CAT activity). A similar protocol was followed as above except the nanoceria samples were not stored in 33% Biotene:67% TRIS over night, they were diluted directly from storage in water to the plate at 300 μM Ce and the photo was taken within 10 minutes of the onset of the assay at 37° C.

### Results

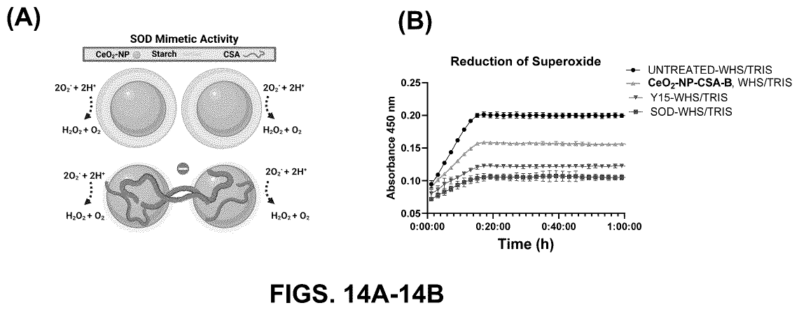

Both single nanoceria [Y-15, starch coating] vs CeO₂—NP-CSA-B demonstrated superoxide dismutase (SOD) activity (FIG. 14A). Plate based assay (SOD kit, Millipore Sigma) in 33:67 WHS/Assay buffer at 10 μM Ce demonstrated activity compared to SOD enzyme itself (red) using IRB Protocol #2023-0562 (FIG. 14B).

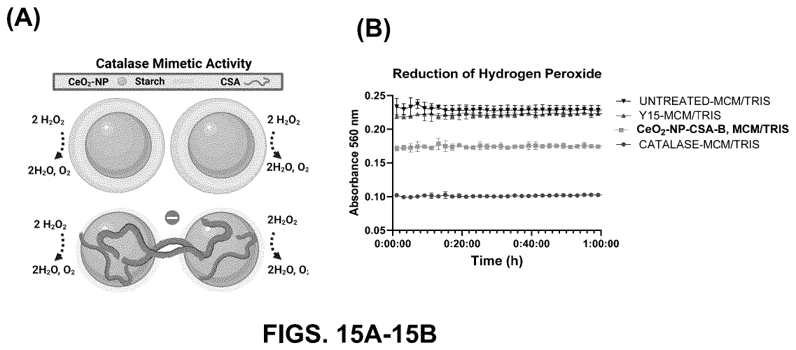

Both single nanoceria [Y-15, starch coating] vs CeO₂—NP-CSA-B demonstrated catalase (CAT) activity (FIG. 15A). Plate based assay (Amplex Red Assay, Invitrogen) was used to screen CAT activity in 33:67 MCM/TRIS buffer pH 7.0 at 150 μM Ce demonstrated activity compared to CAT enzyme itself (red). MCM (Forsythe Institute Protocol) is an artificial saliva (FIG. 15A).

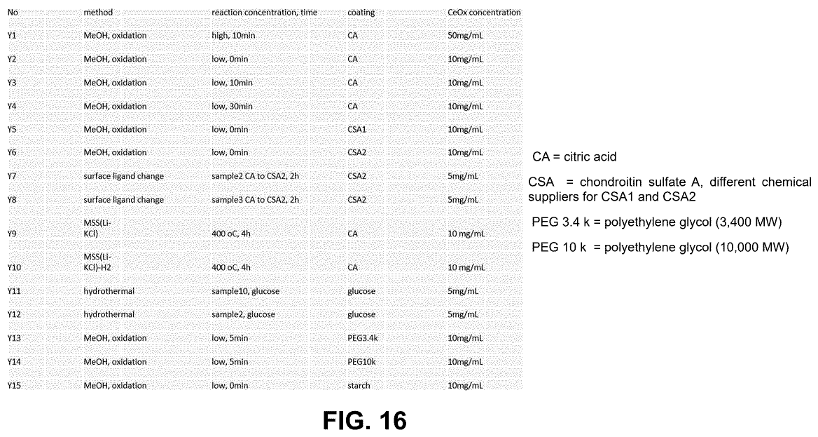

Several different polymer coatings were evaluated with respect to SOD and CAT activity (FIG. 16). FIGS. 17A-17B show SOD and CAT activity for the different polymer coatings. Inhibition comparison of the top nanoceria formulations on going from TRIS buffer (pH 7.0) (FIG. 17A) to a mixture of (33%) WHS (SOD activity) and a mixture of (33%) MCM (CAT activity) (FIG. 17B). CeO₂—NP-CSA-B had the best overall retention of inhibition capacity over both assays upon moving to complex media, despite being a nanohybrid aggregate formulation. MCM was used in the CAT assay due to interferences from WHS in the assay (IRB Protocol #2023-0562).

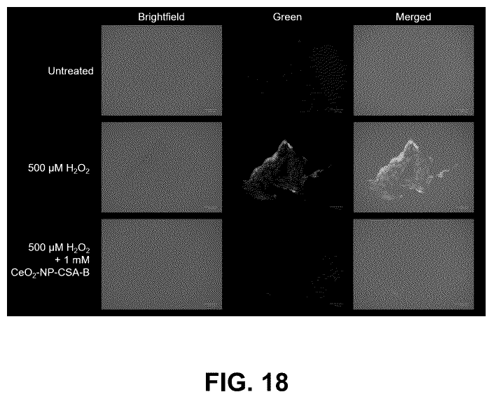

The antioxidant activity of CeO₂—NP-CSA-B with human cells was evaluated. Referring to FIG. 18, human gingival fibroblasts (HGF) were seeded in normal growth media (10% FBS in DMEM) at a density of 15,000 cells per well in a tissue-culture treated 96-well plate and allowed to adhere overnight under normal culture conditions (37° C., 5% CO₂). ROS activity was then induced in the fibroblasts using hydrogen peroxide for 50 min, with the concurrent addition of 1 mM CeO₂—NP-CSA-B. Following ROS induction, fibroblasts were washed three times with PBS, then incubated in 20 μM of DCF-DA for 40 min to show fluorescence induced by oxidative damage. Cells were washed twice with PBS, then imaged under a fluorescence microscope. CeO₂—NP-CSA-B exhibits a strong ability to protect cells from oxidative damage that could occur during periodontitis.

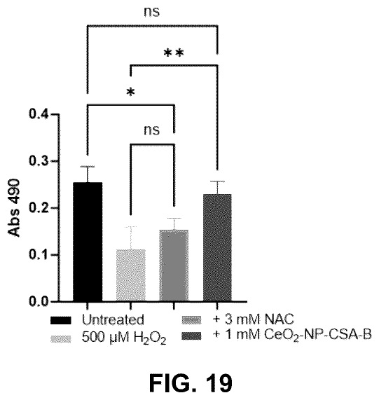

Referring to FIG. 19, human gingival fibroblasts (HGF) were exposed to hydrogen peroxide (and DCF-DA) as described above (FIG. 18) and then were incubated for 2 hours with MTS and optical density was measured with a plate reader spectrophotometer in order to assess viability following ROS induction. CeO₂—NP-CSA-B outcompeted a known and FDA approved antioxidant (N-acetyl cysteine) in protecting cells oral cells from ROS damage. Chronic oxidative damage is linked to common oral pathologies as described previously.

Referring to FIG. 20, human gingival fibroblasts HGFs were seeded into a 96-well tissue culture plate at a density of 15 k cells/well and left to adhere overnight. ROS was induced using 500 μM of H₂O₂, and additional treatments (as specified) were added to the cells and incubated for 1 hour. Following 1 hour treatment, cells were washed, stained with DCF-DA, rinsed and then the MTS assay was performed. The optical density (OD) was read at 490 nm following 1 hour of incubation with MTS reagent. NAC=N-acetyl cysteine, an FDA approved anti-oxidant.

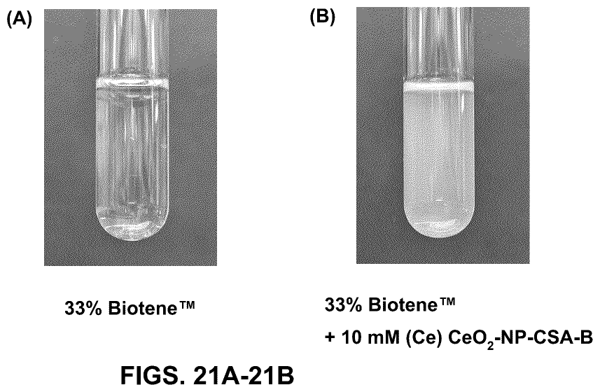

The storage stability of CeO₂—NP-CSA-B in Biotene™. Biotene™ was diluted with MQ water and stored at room temperature for 5 days (FIG. 21A). Biotene™ was spiked with CeO₂—NP-CSA-B and allowed to stand for 5 days at room temperature with minimal mixing (FIG. 21B). The pH of the nanohybrid aggregate mixture at this concentration is 7.0-7.4, similar to that of whole human saliva.

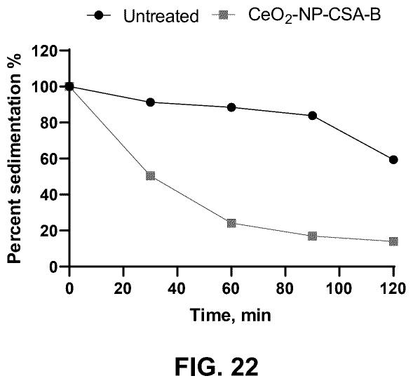

The ability of CeO₂—NP-CSA-B to increase the function of Biotene™ was evaluated. FIG. 22 shows the clearance activity of S. mutans UA159 by CeO₂—NP-CSA-B. S. mutans UA159, a dental caries causing pathogen, was inoculated into a 33% Biotene/67% WHS3 mixture and treated with 1000 μM CeO₂—NP-CSA-B. Sedimentation (clearance) of S. mutans was only found in treated samples as indicated by the loss of OD600 measured by UV-Vis over 2 h.

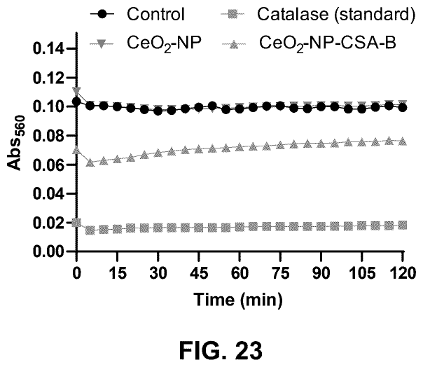

FIG. 23 shows the catalase activity of CeO₂—NP-CSA-B following storage in Biotene™ The Amplex Red (Catalase Assay, Invitrogen) was carried out after storage of CeO₂—NP-CSA-B (#2*) and CeO₂—NP (3-5 nm) separately in 33% Biotene™/67% TRIS buffer (pH 7.0) for ˜24 h at 4 deg. The above assay was carried out in TRIS buffer (pH 7.0) over 2 h at 37° C. at 150 μM Ce. The lower absorbance of sample with CeO₂—NP-CSA-B at 560 nm indicates higher catalase activity, superior to single CeO₂—NP (3-5 nm) which was inactive under the above conditions following storage.

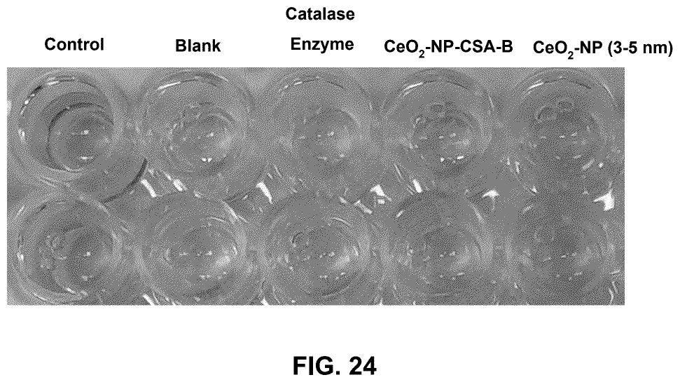

FIG. 24 shows the Amplex Red (Catalase Assay, Invitrogen) was carried out comparing CeO₂—NP-CSA-B to single CeO₂—NP (3-5 nm) separately in 33% Biotene™/67% TRIS buffer (pH 7.0). Photos taken within minutes of adding the indicator. Reduced pink color is associated with higher catalase activity as seen with the catalase enzyme itself and CeO₂—NP-CSA-B. Both CeO₂—NP-CSA-B and CeO₂—NP were carried out at 300 μM Ce.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A coated nanoparticle aggregate comprising aggregated hydrolyzed tetravalent metal salt nanoparticles, wherein the aggregated hydrolyzed tetravalent metal salt nanoparticles comprise a coating of a pharmaceutically acceptable salt of a glycosaminoglycan.

2. The coated nanoparticle aggregate of claim 1, wherein the hydrolyzed tetravalent metal salt comprises a hydrolysis product of a Ce(IV) salt, a Zr(IV) salt, a Hf(IV) salt, or a Ti(IV) salt.

3. The coated nanoparticle aggregate of claim 1, wherein the hydrolyzed tetravalent metal salt comprises a hydrolysis product of Ce(IV) salt.

4. The coated nanoparticle aggregate of claim 1, wherein the hydrolyzed tetravalent metal salt comprises CeO₂.

5. The coated nanoparticle aggregate of claim 1, wherein the aggregated hydrolyzed tetravalent metal salt nanoparticles have an average microscopic diameter of about 20 nm to about 500 nm.

6. The coated nanoparticle aggregate of claim 1, wherein the glycosaminoglycan comprises a sulfated glycosaminoglycan.

7. The coated nanoparticle aggregate of claim 1, wherein the glycosaminoglycan comprises heparin, heparan sulfate, chondroitin sulfate, keratan sulfate, hyaluronic acid, or any combination thereof.

8. The coated nanoparticle aggregate of claim 1, wherein the coating comprises the sodium salt of chondroitin sulfate

9. The coated nanoparticle aggregate of claim 1, wherein the coated nanoparticles further comprise one or more additional metal salts of Ca(II), Sr(II), Ba(II), Zn(II), Cu (II), Be(II), Ni(II), Fe(II), Co(II), Mn(II), Cr(II), V(II), Ti(II), Sc(II), Cd(II), Hg(II), cacodylic acid (As) sodium salt, or any combination thereof.

10. The coated nanoparticle aggregate of claim 1, wherein the coated nanoparticle aggregate is produced by the process comprising admixing the aggregated hydrolyzed tetravalent metal salt nanoparticles with the pharmaceutically acceptable salt of chondroitin sulfate in a solvent.

11. A pharmaceutical composition comprising the coated nanoparticle aggregate of claim 1 and a pharmaceutically acceptable carrier.

12. The composition of claim 11, wherein the composition comprises an oral or topical composition.

13. The composition of claim 11, wherein the composition comprises a mouthwash comprising sodium monofluorophosphate, sodium fluoride, or a combination thereof.

14. A method for reducing or preventing the formation of a biofilm in a subject, the method comprising administering to the subject the coated nanoparticle aggregate of claim 1.

15. The method of claim 14, wherein the coated nanoparticle aggregate reduces or prevents the formation of the biofilm in an oral cavity of the subject.

16. A method for reducing or preventing oxidative damage or stress of a tissue in a subject, the method comprising administering to the subject the coated nanoparticle aggregate of claim 1.

17. The method of claim 16, wherein the coated nanoparticle aggregate reduces or prevents oxidative damage in or around the oral cavity of the subject.

18. A method for reducing or preventing the formation of a plaque, dental caries, or a periodontal disease in a subject, the method comprising administering to the subject the coated nanoparticle aggregate of claim 1.

19. A method for treating or preventing skin cancer or oral cancer in a subject, the method comprising administering to the subject the coated nanoparticle aggregate of claim 1.

20. A medical device comprising one or more surfaces partially or completely coated with the coated nanoparticle aggregate of claim 1.
